(19) United States
(12) Patent Application Publication
(10) Pub. No.: US 2013/0040289 A1
(43) Pub. Date: Feb. 14, 2013

(54) BREAST MILK ETHANOL SCREENING SYSTEM AND METHOD

(71) Applicant: Upspring Ltd., Austin, TX (US)

(72) Inventors: Julie JUMONVILLE, Perth (AU); Elizabeth SMITH, Austin, TX (US); Matthew POPE, Austin, TX (US)

(73) Assignee: Upspring Ltd., Austin, TX (US)

(21) Appl. No.: 13/654,312

(22) Filed: Oct. 17, 2012

Related U.S. Application Data
(62) Division of application No. 12/568,649, filed on Sep. 28, 2009, now abandoned.
Application No. 12/568,649 is a continuation-in-part of application No. 11/361,590, filed on Feb. 24, 2006, now abandoned.

(51) Int. Cl. G01N 21/78; G01N 33/53; G01N 27/26; C12Q 1/28

(52) U.S. Cl. 435/5; 435/28; 435/7.93; 436/501; 436/23

(57) ABSTRACT

A test kit detects the presence of a target analyte in a fluid sample. In particular, the test kit includes reagents capable of detecting the target analyte of interest in breast milk. More particularly, the test kit is capable of detecting the presence of alcohol, caffeine, nicotine, drugs of abuse, therapeutic drugs, triglycerides, lactose, capsaicin, and gluten, for example, in breast milk.

### CROSS-REFERENCES TO RELATED APPLICATIONS

This application is a divisional application of co-pending U.S. patent application Ser. No. 12/568,649, filed Sep. 28, 2009 and titled “Breast Milk Ethanol Screening System and Method”, which is a continuation-in-part of U.S. patent application Ser. No. 11/361,590, filed Feb. 24, 2006 and titled “Breast Milk Ethanol Screening System and Method”, now abandoned, the disclosures of which are expressly incorporated herein by reference in their entirety.

### BACKGROUND OF THE INVENTION

1. Field of the Invention

The invention generally relates to screen testing and, more particularly, relates to screening the breast milk of lactating females prior to breastfeeding for a target analyte.

2. Related Art

There are a variety of substances consumed by a mother that pass readily into her breast milk at levels reaching concentrations similar to those found in maternal blood. Although the amount of the substance(s) that the nursing infant is exposed to is typically small, the substances can still affects to the nursing infant, such as altering sleeping patterns. Such substances include, for example, alcohol, caffeine, nicotine, gluten, lactose, pathogens, therapeutic drugs, and drugs of abuse (“target analytes”).

Various dangers of excessive ethanol alcohol consumption by humans, typically in the form of alcoholic beverages, are known and publicized. The predominant focus of research and reporting has typically been the effects of such consumption on the particular consumer. Of course, health concerns, legal requirements and restraints, and other similar issues have often been raised, particularly in heavy alcohol consumers.

Research of alcohol consumption by mothers during pregnancy, and impact to the in utero infant, has received much attention. Less attention has tended to be directed, however, to post-partum consumption of alcohol by breast-feeding mothers. In recent years, there have been numerous studies performed in the areas of breast milk composition, nutritional values of breast-feeding, post-birth maternal alcohol use and effects on lactation and hormones involved in breast-feeding, and maternal alcohol use and its effects on infant development and behavior. Some of these studies indicate negative effects for the infant at alcohol levels that start at 30 mg/dL.

Presently, it is widely held that human milk is uniquely superior to alternatives for infant feeding, and breast-feeding generally appears quite beneficial for both the mother and the infant. Breastfeeding and the Use of Human Milk, American Academy of Pediatrics, Work Group on Breastfeeding (1997). Of course, breast-feeding presents concerns for the mother, in that dietary, intake, drug, and other effects can be passed through to the feeding infant. This can constrain breast-feeding mothers from normal social lifestyles and require various dietary and activity constraints or practices. It also can cause consternation and worry to the mother. One major concern of breast-feeding mothers is whether or not alcohol consumption by the mother can adversely or otherwise affect the infant.

Folklore and traditional wisdom (prior to at least as recent as about 1998, and even continuing to an extent thereafter until very recently) has been that some alcohol consumption, particularly of beer and wine, by breast-feeding mothers may be beneficial for increased lactate production, initiation of breastfeeding, infant nutrition, and other enhancement of breastfeeding success. Alcohol's Effect on Lactation, Alcohol Res. Health, 25(3):230-4 (Mennella, et al., 2001); Beer and Breastfeeding, Adv. Exp. Med. Biol., 478:23-8 (Koletko, et al., 2000); Breastfeeding & Drugs: Alcohol, http://www.breastfeedingbasics.org/cgi-bin/deliver.cgi/content/Drugs/alcohol.html (O'Connor, 1998)(moderate alcohol use on infant development is inconclusive; occasional alcoholic drink by lactating woman probably fine); Alcohol and breastfeeding, J. Hum Lact., 11(4); 321-323 (Anderson, 1995) (alcohol believed to stimulate breast milk production; may stimulate sucking initially); Infants' suckling responses to the flavor of alcohol in mothers' milk, Dev. Psychobiol., 26(8):459-66 (Mennella, 1993); Transfer of alcohol to human milk, New Eng. J. Med., 325(14):981-985 (Mennella, et al., 1991) (limited amount of alcohol believed transferred to mothers' milk); Beer, breastfeeding, and folklore, N. Engl. J. Med, 321(7):425-30 (Menella, et al., 1989).

Studies now show that alcohol presence in mothers' milk at certain levels may, in fact, have significant effects on breast-feeding and breast-fed infants. For example, infants may nurse more frequently but consume significantly less breast milk if it contains alcohol [Beer, breastfeeding, and folklore, N. Engl. J. Med., 321(7):425-30 (Menella, et al., 1989)]; infant motor development may be adversely affected if exposed to breast milk alcohol [Alcohol, Breastfeeding, and Development at 18 Months, Pediatrics 2002; 209; 72 (Little, et al., 2002); Maternal alcohol use during breast-feeding and infant mental and motor development at one year, N. Engl. J. Med., 322(5):338-9 (Little, et al., 1990)]; lactate production by drinking mothers is not increased, and may decrease [Lactation and alcohol: clinical and nutritional effects, Arch. Latinoam Nutr., 54(1):25-35 (de Araujo Burgos, et al., 2004); Alcohol's Effect on Lactation, Alcohol Res. Health, 25(3):230-4 (Mennella, 2001)]; infant sleep patterns may change with intake of breast milk containing alcohol [Sleep disturbances after acute exposure to alcohol in mothers' milk, Alcohol, 25(3):153-8 (Mennella, et al., 2001); Effects of Exposure to Alcohol in Mother's Milk on Infant Sleep, Pediatrics, 101(5):e2 (Menella, et al., 1998)]; and other problems to the infant and/or the mother.

In light of these new indications (observed primarily only since after about of 1989 and more so in even more recent years in the 21^{st} century) of potential harms of alcohol in breast-feeding, it appears advantageous to avoid breast-feeding if alcohol is present in breast milk. Authorities indicate that time studies on time to zero level of alcohol in breast milk are dependent on a number of factors, particularly including the amount of alcohol consumed by and the body weight of the mother. Alcohol and Breast Feeding: Calculation of Time to Zero Level in Milk, Biol. Neonate, 80:219-22 (Ho, et al., 2001) (nomogram of weight and number of drinks to estimated time to zero from this source is provided in Appendix A). These same authorities recommend that the breast-feeding mother try to delay breast feeding to allow complete elimination of alcohol from her breast milk. Id. Notwithstanding the time to zero estimates presently available (as in the foregoing source), that source recognizes that the values may be faster or slower in some women, depend on many factors beyond weight and amount of alcohol consumed, and can only be estimated based on average results in the studies.

For at least 20 years or so (i.e., since as early as about 1985), various tests and test kits for individual use have been proposed for testing alcohol levels in saliva and urine. More accurate measures of alcohol concentrations in the body after consuming ethanol alcohol beverages, however, generally require clinical diagnostic blood tests. The tests and test kits for individual use have been limited to determination of bodily alcohol levels to avoid illegal driving and the like. There have not been any individual-use alcohol tests or test kits for purposes of gauging for breastfeeding. Of course, breast milk levels of alcohol may not be well reflected from saliva, urine, and/or blood tests, as breast milk is produced through bodily processes of the mother that can significantly differ from the bodily processes for saliva, urine, and blood.

It would therefore be advantageous to provide individual-use tests and test kits for directly detecting a target analyte that may be presence in breast milk. Moreover, levels of a target analyte contained in breast milk may/may not be important to the ramifications of breast-feeding. Therefore, it would be advantageous to provide such tests and test kits that detect one or more target analytes. The American Academy of Pediatrics recommends exclusive breast feeding for six months, therefore tests that detect certain analytes in breast milk can extend the life of breast feeding by allowing mothers to return to a more normal social life and by allowing mothers to test for substances that she might be concerned with passing to their child through breast-feeding, for instance. Of course, compositional levels of a target analyte in breast milk could also be considered important for the determinations, in certain instances. So, such tests and test kits could additionally/alternatively beneficially provide for a target analyte level determinations in the milk. Objectives of individual-use tests and test kits in these regards could provide substantial health and other benefits.

It would, therefore, be a significant improvement in the art and technology to provide tests and test kits for detection of one or more target analytes in breast milk of breast-feeding mothers. The invention provides numerous advantages and improvements, including improvements and nuances in the foregoing respects.

### BRIEF SUMMARY OF THE INVENTION

According to one aspect of the invention a breast milk lactating kit may include a test device including at least one of a sampling portion, a single chemistry portion, or a multiple chemistry portion, and a target analyte test reagent disposed on at least one of the sampling portion, the single chemistry portion, or the multiple chemistry portion, where the target analyte may be reactive with target analyte in breast milk, an oxidation-protected package containing at least a portion of the test device, and an output device to indicate the presence, absence, and/or concentration of the target analyte. The package may be a single-use package such as a pouch or a multiple-use package such as a container or a re-sealable pouch including a desiccant.

The target analyte test reagent may be a alcohol test reagent. The alcohol test reagent may include a hydrogen donor indicator, an alcohol oxidase, and a peroxidase. The hydrogen donor indicator may include ABTS (2,2′-azinobis(3-ethylbenzothiazoline-6-sulfonic acid)), a mixture of 4-aminoantipyrine (AAP) with 4-hydroxybenzosulfonate, a mixture of AAP with phenol, 3,3′,5,5′-tetramethylbenzidine (TMD) tetramethylbenzidine, and a mixture of potassium iodide and starch. The hydrogen donor may be a mixture of potassium iodide and starch.

The target analyte test reagent may include one or more reagents for performing an immunoassay. The target analyte may include alcohol, nicotine, gluten, lactose, drugs of abuse, therapeutic drugs, triglycerides, and capsaicin.

The device may be a strip such as an enzymatic test strip. The device may also include a handling portion. The multiple chemistry portion may be reactive with multiple target analytes. The chemistry portion may include multiple test devices and the at least one sampling portion may include one of a common sampling portion connected to each of the test devices or individual sampling portions, each connected to one of the test devices. The device may also include a wicking portion containing stop line chemistry.

The test kit may also include a viewable alcohol concentration standard device. The standard device may be a card having a plurality of color tabs such that each of the plurality of the color tabs is a different color where each color corresponds with a different alcohol concentration level. Alternatively, the display may indicate a value that is indicative of a change in electrical activity of the single or multiple chemistry portions, such as a change in potential, voltage and/or current.

According to a further aspect of the invention, a system for determining the absence, presence, or concentration of a target analyte in breast milk lactate may include a test device, at least one of a sampling portion, a single chemistry portion, or a multiple chemistry portion, and a target analyte test reagent disposed on at least one of the sampling portion, the single chemistry portion, or the multiple chemistry portion, and an analysis device for determining the absence. Presence, or concentration of the target analyte in the breast milk lactate.

The target analyte test reagent may be an alcohol test reagent. The alcohol test reagent may react with alcohol and after a predetermined period of time has lapsed, the alcohol induces a detectable change in said alcohol test reagent. The detectable change may be a color change in said alcohol test reagent. The analysis device may include a card having a plurality of color tabs such that each of the plurality of color tabs corresponds with a different alcohol concentration level.

The detectable change may be a change in electrical properties such as a change in current, a change in potential, or a change in current. Specifically, the change in electrical properties may be a change in current.

The target analyte test reagent may include one or more reagents for performing an immunoassay. The target analyte may include alcohol, nicotine, gluten, lactose, drugs of abuse, therapeutic drugs, triglycerides, and capsaicin.

In yet another aspect of the invention, a method of protecting a nursing infant from exposure to a target analyte present in breast milk that may have adverse effects on the nursing infant may include obtaining a breast milk sample from a subject, where the subject is suspected of ingesting a target analyte that may be harmful to the nursing infant, detecting the presence, absence, or concentration of the target analyte present in the breast milk by detecting an observable change in at least one reagent, wherein if the change indicates the presence or concentration of the target analyte above a predetermined level, waiting a period of time to allow for clearance of the target analyte from the breast milk prior to breast feeding. The observable change may be a colormetric change.

The one or more reagents may include one or more reagents for performing an immunoassay. The one or more reagent may include reagents for performing a multiple enzyme test. The one or more reagent may include reagent for performing a single enzyme test. The target analyte may include lactose, gluten, nicotine, drugs of abuse, therapeutic drugs, triglycerides, capsaicin, and alcohol.

The observable change may be a change in electrical properties, such as a change in potential, a change in voltage, or a change in current. Specifically, the change in electrical properties is a change in current.

According to another aspect of the invention a method of determine if a lactating mother is afflicted with mastitis may include providing a test strip including a sampling portion, and one or more reagents that can detect a target analyte associated with mastitis disposed on at the sampling portion, contacting the one or more reagents on the sampling portion with the breast milk lactate for a predetermined period of time to allow any target analytes associated with mastitis present in the breast milk lactate to react with the test reagent and induce a detectable change in the test reagent, and observing a result after the predetermined period of time has lapsed wherein a detectable change in the target analyte test reagent is indicative of the presence of the target analyte associated with mastitis in the breast milk lactate and is indicative of mastitis.

The detectable change in the test reagent may be a color change. The target analyte associate with mastitis concentration may be estimated in the breast milk lactate by comparing the visible color change in the test reagent to a viewable concentration standard device. The target analyte associate with mastitis concentration may be estimated in the breast milk lactate by comparing the visible color change in the test reagent to a viewable concentration standard device. The reagents may comprise reagents for performing an immunoassay. The reagents may comprise reagents for performing a dual-enzyme test. The target analyte associated with mastitis may include leukocytes, inflammatory cytokines, salt, bacterial waste products, and other substances that become known or are known in the art.

The observable change may be a change in electrical properties, such as a change in potential, a change in voltage, or a change in current. Specifically, the change in electrical properties is a change in current.

According to one aspect of the invention, a method of testing breast milk lactate for the presence of alcohol may include providing a test strip including a handling portion, at least one of a sampling portion, a single secondary chemistry portion, or a multiple secondary chemistry portion, and an alcohol test reagent disposed on at least one of the sampling portion, the single secondary chemistry portion, or the multiple secondary chemistry portion contacting the alcohol test reagent on the sampling portion with the breast milk lactate for a predetermined period of time to allow any alcohol present in the breast milk lactate to react with the alcohol test reagent and induce a detectable change in the alcohol test reagent, and observing a result after the predetermined period of time has lapsed wherein a detectable change in the alcohol test reagent is indicative of the presence of alcohol in the breast milk lactate.

The detectable change in the alcohol test reagent may be a color change. The alcohol concentration may be estimated in the breast milk lactate by comparing the visible color change in the alcohol test reagent to a viewable concentration standard device. The viewable concentration standard device may include a card having a plurality of color tabs such that each of the plurality of color tabs is a different color corresponding to a different alcohol concentration level.

The observable change may be a change in electrical properties, such as a change in potential, a change in voltage, or a change in current. Specifically, the change in electrical properties is a change in current.

The sampling portion may include a carrier vehicle that retains the alcohol test reagent. The carrier vehicle may be fabricated from cotton. The alcohol test reagent in the contacting step may include a hydrogen donor indicator, an alcohol oxidase, and a peroxidase. The hydrogen donor may include ABTS (2,2″-azinobis(3-ethylbenzothiazoline-6-sulfonic acid)), a mixture of 4-aminoantipyrine (AAP) with 4-hydroxybenzosulfonate, a mixture of AAP with phenol, 3,3′,5,5′-tetramethylbenzidine (FMB) tetramethylbenzidine, and a mixture of potassium iodide and starch. The hydrogen donor may be a mixture of potassium iodide and starch.

The predetermined period of time in said contacting step may be about 2 minutes. The test strip may be for one-time general consumer use and is disposable. The handling portion of the testing strip may be handled by an individual without hand contact of the sampling portion.

Additional features, advantages, and embodiments of the invention may be set forth or apparent from consideration of the following detailed description, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention, are incorporated in and constitute a part of this specification; illustrate embodiments of the invention and together with the detailed description serve to explain the principles of the invention. No attempt is made to show structural details of the invention in more detail than may be necessary for a fundamental understanding of the invention and various ways in which it may be practiced.

FIG. 1 illustrates a perspective view of one embodiment of a test device for detecting presence of alcohol in breast milk of a breast-feeding mother, according to principles of the invention.

FIG. 2 illustrates a side view of the test strip of FIG. 1, including a carrier strip and a reactive agent on the carrier strip.

FIG. 3 illustrates a side view of an alternate embodiment of a test strip of the invention, similar to that of FIG. 1 but having a pool drop of reactive agent on an end of the carrier strip.

FIG. 4 illustrates a side view of another one embodiment of test strip, including reactive agent affixed to an end of the carrier strip.

FIG. 5 illustrates a side view of another embodiment of test strip, including a carrier strip that is impregnable or otherwise carries interstitially therewith a reactive agent.

FIG. 6 illustrates a side of yet another one embodiment of test strip, the test strip including a carrier vehicle at an end of the strip, the carrier vehicle serving to absorb or hold a reactive agent.

FIG. 7 illustrates a perspective view of an embodiment of the test kit, including the test strip of FIG. 1, the test kit having a sealed enclosure pack containing the test strip and a desiccant.

FIG. 8 illustrates a front view of an alternate embodiment of a test kit, having a sealed enclosure containing a reactive agent of a carrier strip, with a handling portion of the carrier strip protruding from within the sealed enclosure.

FIG. 9 illustrates a perspective view of an embodiment of a bottle test kit, including a vial and cap, the cap having a test piece extending there through with a reactive agent of the test piece contained within the vial and cap enclosure.

FIG. 10 illustrates a perspective view of another embodiment of a bottle test kit, a vial and cap enclosure containing a reactive agent, a separate handling strip is provided and includes or is formed of a material to carry the reactive agent when dipped into the vial into the agent.

FIG. 11 illustrates a side perspective view of another embodiment of a test kit, including an enclosed packet of reactive agent, the enclosed packet being semi-permeable to permit soaking of the reactive agent contents with breast milk applied thereto, and providing for externally visible observance of test results with the reactive agent.

FIG. 12 illustrates a method for testing a breast milk lactate for presence of alcohol, using a test device, according to principles of the invention.

FIG. 13 illustrates one embodiment of a lateral flow testing device for detecting the presence, absence, or concentration of an analyte in a fluid sample according to principles of the invention.

FIG. 14 illustrates one embodiment of a lateral flow testing device including multiple testing strips for detecting the presence, absence, or concentration of multiple analytes in a fluid sample according to principles of the invention. Panel I depicts an internal view of the device and Panel II depicts an external view of the device.

FIG. 15 illustrates one embodiment of a lateral flow testing device including multiple testing strips having one common sampling region for detecting the presence, absence, or concentration of multiple analytes in a fluid sample according to principles of the invention. Panel I depicts an internal view of the device and Panel II depicts an external view of the device.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “an analyte” is a reference to one or more analytes and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law.

Accordingly, provided immediately below is a “Definition” section, where certain terms related to the invention are defined specifically for clarity, but all of the definitions are consistent with how a skilled artisan would understand these terms. Particular methods, devices, and materials are described, although any methods and materials similar or equivalent to those described herein can be used in the practice or testing of the invention. All references referred to herein are incorporated by reference herein in their entirety.

The term “target analyte,” “analyte,” or “analyte of interest,” as used herein is meant any molecule, compound, biomolecule, or particle to be detected in a fluid sample. Analyte may include biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores. Analytes also include caffeine; nicotine; capsaicin, gluten, lactose, triglycerides, leukocytes, sodium, and therapeutic and abused drugs.

“Immunoassays,” “ELISA,” “enzyme-immunoassay,” or “EIA,” as used herein, generally refers to a technique to detect the presence of an antibody or target analyte in a sample. Those of ordinary skill in the art appreciate that ELISA is widely-used method for measuring the concentration of a particular target molecule (e.g., alcohol, caffeine, drug of abuse, or prescription drug) in a fluid such as breast milk. In one aspect, the test requires the antibodies be affixed to a solid surface and a preparation of the same antibodies couple to an enzyme that is capable of producing an observable result, such as a color change. Generally, the ELISA assay involves contacting the immobilized antibodies with the fluid (e.g., breast milk) to be assayed. Any target analytes present in the fluid will bind to the immobilized antibodies. An antibody-enzyme conjugate is added to the reaction mixture. After a set interval, the colored product is formed. The intensity of color is directly proportional to the concentration of bound target analyte to the immobilized antibodies. Conversely, the ELISA may also be used to detect the quantity of a specific antibody in a fluid. Here, the appropriate antigen is immobilized and contacted by a fluid possibly containing the antibodies. After a particular time interval, an enzyme-conjugated anti-immunoglobulin that is conjugated to an enzyme capable of producing an observable result (e.g., color change) is added. The intensity of color produced is proportional to the amount of enzyme-labeled antibodies bound, which in turn, is proportional to the concentration of the antibodies being assayed.

The term “label,” as used herein, refers to a detectable compound or composition which can be conjugated directly or indirectly to the antibody, or the target analyte of the invention. The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label may be a specific binding molecule which itself may be detectable (e.g., biotin, avidin, streptacidin, digioxigenin, maltose, oligohistidine, 2,4-dinitrobenzene, phenylarsenate, ssDNA, dsDNA, and the like).

The term, “synthetic molecules,” “synthetic receptors,” or “artificial enzymes,” as used herein generally refers to molecules that display enzyme-like characteristics, but have their own set of unique characteristics as well. Whereas enzymes have a higher level of specificity than catalysts in general, synthetic receptors often offer an even higher level due to the customized chemistry and a more specific bonding affinity. A synthetic receptor is essentially a high molecular weight organic molecule that has been prepared with chemistry specific bonding sites that mimic a reaction similar to that found in enzymatic chemistry. On these molecules, the chemical reactions occur with high selectivity and rate in a small part of the macromolecule known as the active site. The chemistry at these sites can be tailored to react in any of the following ways: (i) Sequestering: The chemistry at the active site has a strong bonding affinity for a given species in the matrix. As the chemistries are attracted by bonding forces and fields, they pull the specific species from the matrix and form a direct bond with it, essentially ‘latching’ onto it and making it part of the overall macromolecule, and (ii) Displacement: The chemistry at the active site has a lower bonding affinity with the macromolecule at the given region than a given species from the matrix. As the targeted species from the matrix nears these regions on the macromolecule, there is essentially a ‘changing of guards’ at the active site where the originally bonded chemistry is displaced into the matrix and the targeted species is now bound to the macromolecule.

“Lateral flow test,” as used herein generally refers to a device used to detect the presence (or absence) of a target analyte in a fluid sample, such as breast milk. In general, lateral flow tests may be in the form of an immunoassay, enzymatic, artificial receptor, or dye indicator test in which the test sample reacts with the indicator reagents and flows along a solid substrate via capillary action to a viewable results region. For example, an immunoassay lateral flow test may be carried out as follows. After the fluid sample is applied to the test it encounters a colored reagent which mixes with the sample and transits the substrate encountering lines or zone which have been pretreated with an antibody or antigen. Depending upon the analyte present in the fluid sample, the colored reagent can become bound at the test line or zone. Lateral flow tests may operate as either competitive or sandwich assays. The sandwich method is carried out in the following manner. The sample first encounters colored particles which are labeled with antibodies raised against the target analyte. The test line will also contain antibodies to the same target, although it may bind to a different epitope on the analyte. The test line will show as a colored band in positive fluid samples. The competitive assay may be implemented as follows. The fluid sample encounters colored particles which are labeled with the target analyte or an analogue. The test line contains antibodies to the target/its analogue. Unlabeled analyte in the fluid sample will block the binding sites on the antibodies preventing uptake of the colored particles. The test line will show as a colored band in negative samples.

“Antibodies,” and “antibody fragments thereof,” as used herein generally refers to antibodies, digestion fragments, specified portions and variants thereof, including antibody mimetics or comprising portions of antibodies that mimic the structure and/or function of an antibody or specified fragment or portion thereof, including single chain antibodies and fragments thereof. The invention encompasses antibodies and antibody fragments capable of binding to a target analyte, such as alcohol, caffeine and any of its metabolites, drugs of abuse and their metabolites, prescription drugs and their metabolites, over the counter drugs and their metabolites, and capsaicin. Antibodies suitable for use in this invention are obtained by techniques well known in the art. For instance, polyclonal antibodies are obtained by immunizing a species of animal that differs from the species producing the antigen. For example, monoclonal antibodies may be obtained by fusing the splenocytes of an immunized animal with a plasmacytoma cell line by the addition of polyethylene glycol to the cell mixture, thereby forming hybridoma cells which are suspended and then plated to tissue culture plates. Only the cultures producing antibodies that are immunologically reactive with antigen are cloned. See, e.g., U.S. Pat. No. 4,376,110, hereby incorporated herein by reference.

The term “antibody fragment,” as used herein, refers to a portion of a full length antibody, generally the antigen binding or variable domain thereof. Specifically, for example, antibody fragments may include Fab, Fab′, F(ab′)₂, and Fv fragments; diabodies; linear antibodies; single-chain antibody molecules; and multispecific antibodies from antibody fragments.

The term “monoclonal antibody,” as used herein generally refers to an antibody obtained from a population of substantially homogeneous antibodies, i.e., the individual antibodies comprising the population are identical. Monoclonal antibodies are highly specific, being directed against a single antigenic site. In contrast to polyclonal antibody preparations, which typically include different antibodies directed against different epitopes, each monoclonal antibody is directed against a single epitope on the antigen. The modifier “monoclonal” merely refers to the character of the antibody and is not to be construed as requiring production of the antibody by any particular method. Specifically, for example, monoclonal antibodies may be made by hybridoma methodologies, or may be made by recombinant DNA methods, or may be isolated from phage antibody libraries using known techniques.

The term “antigen,” as used herein, generally refers to a substance that is capable, under appropriate conditions, of inducing a specific immune response and of reacting with the product of that response, such as an antibody.

The term “specificity,” as used herein, generally refers to the ability of an individual antibody combining site to react with only one antigenic determinant or the ability of a population of antibody molecules to react with only one antigen. In general, there is a high degree of specificity in antigen-antibody reactions. Antibodies can distinguish differences in (i) the primary structure of an antigen, (ii) isomeric forms of an antigen, and (iii) secondary and tertiary structure of an antigen. Antibody-antigen reactions that exhibit high specificity exhibit low cross reactivity.

The term “biomolecule,” as used herein generally refers to biologically derived molecules such as cells, peptides, small polypeptides, long polypeptides, proteins, antigens, antibodies, tagged proteins, oligonucleotides, nucleotides, polynucleotides, aptamers, DNA, RNA, carbohydrates, etc., and complexes thereof.

The term “fluid sample,” as used herein generally refers to any biological fluid such as saliva, breast milk, urine, blood, placenta, tears, plasma, cerebrospinal fluid, amniotic fluid, and serum.

Compositions, methods, devices, and testing kits for determining the presence and concentration of a target analyte in a fluid sample such as breast milk are disclosed herein. More particularly, a testing device for assaying a fluid sample such as breast milk for a target analyte by using an indicator reagent composition is disclosed herein. The indicator reagent composition undergoes a detectable and measurable response upon contact with the fluid sample containing the target analyte of interest. The indicator reagent screening assays that are suitable for the invention include those known in the art such as, dual enzyme-based systems such as the oxidases/peroxidase systems, single enzyme-based systems, and ELISA assays, for example.

Certain substances consumed by lactating mothers pass readily into her breast milk at levels reaching concentrations similar to those found in maternal blood. Although the amount a nursing infant is exposed to is only a small portion of what the mother ingests, infants may metabolize the substances at different rates than an adult or may not be able to metabolize the substance at all due to decreased liver activity, for example. This may result in affects to the nursing infant, such as an alteration in sleep patterns. Accordingly, testing kits for screening for the presence of a target analyte, can help the mother be sure of complete elimination of the substance prior to breast feeding and preventing or reducing neonatal exposure.

For example, it may be desirable to test breast milk for the presence of nicotine, drugs of abuse, therapeutic drugs, caffeine, capsaicin, lactose, gluten, pathogens, and other biomolecules. Nicotine and its metabolite, continine, readily passes into breast milk. Several studies have shown that nicotine may cause a decrease in basal prolactin production.

Caffeine is a naturally occurring CNS stimulant and is present in many foods and drinks. It is important to test for the presence of caffeine in breast milk prior to feeding because the half-life of caffeine is 4.9 hours in adults and the half-life of caffeine in neonates may be as high as 120 hours. The average cup of coffee contains 100 to 150 mg of caffeine depending on country of origin. Peak levels of caffeine are present in breast milk 60-120 minutes after ingestion. However, each woman's body is unique and metabolizes caffeine at different rates so there is not an exact science or timing on when caffeine is no longer in their system.

It is also desirable to screen breast milk for substances that contribute to colic symptoms such as capsaicin, which is an alkaloid derived from peppers from the Solanaceae. Other substances that have been linked to causing colic symptoms include lactose, gluten, and cruciferous vegetables.

The screening assays of the invention can also be used to determine if the lactating mother is afflicted with mastitis. Mastitis is defined as the presence of inflammation in breast tissue, specifically in the milk-forming portions of the breast. If mastitis is identified early enough the mother can use heat compresses and position the baby to relieve the clogged milk duct before the early onset of mastitis requires the mother to need and antibiotic to clear up the infection. Treatment with antibiotics is controversial, and consensus is that it should be reserved for only the most serious cases, including those with specific laboratory findings associated with bacterial infections. Many inflammatory markers are elevated both in blood and in breast milk during mastitis, whether of bacterial or other origin. Elevations of milk white blood cells (WBC), sodium, and levels of certain inflammatory cytokines have all been reported in women with clinical signs of mastitis. There are several possible approaches to the development of a screening test, which include the detection of white blood cells (WBC), bacterial waste products, specific bacterial markers, and various other markers of inflammation such as carboxypeptidases, angiotension converting enzyme (ACE, neutral endopeptidase, C1 inhibitor; histamines, P selectin, E selectin, IFN γ, IL-8, leukotriene B4, nitric oxide (NO), prostaglandins, TNFα, IL-1, and integrins.

According to one embodiment of the invention, the target analyte may be detected by employing a direct enzymatic test. During this reaction, a target analyte specific oxidase liberates hydrogen peroxide, which subsequently is used as an oxidant in he couples enzymatic reaction catalyzed by horseradish peroxidase. The principle of target analyte determination by analyte-specific oxidase-peroxidase method is based on the measurement of the dye-product accumulation in peroxidative oxidation of chromogen by H₂O₂ generated from the target analyte in the oxidase reaction. In a specific aspect of the invention, the oxidases may include alcohol oxidase, L-amino acid oxidase, uricase and uric acid oxidase, xanthine oxidase, glycine oxidase, monoamino oxidase, diamine oxidase, D-aspartic acid oxidase, liver aldehyde oxidase, and galactose oxidase.

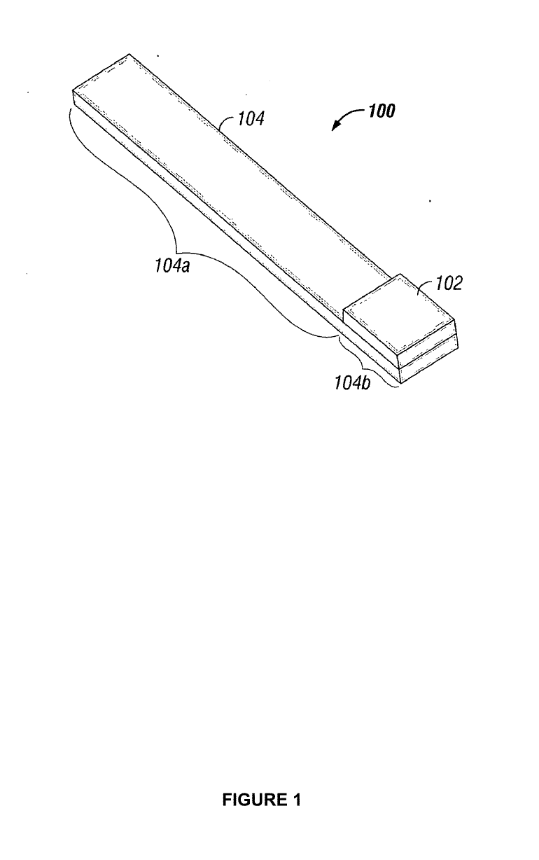

In a specific embodiment, the oxidase is alcohol oxidase. Certain further examples of the alcohol oxidase of the reaction are described, for example, in U.S. Pat. No. 4,430,427. Certain examples of the peroxidase of the reaction, as well as other possible peroxidatively active substances, are described, for example, in U.S. Pat. No. 4,361,648. Of course, other suitable alcohol oxidase enzymes, peroxidases, and indicator agents can be employed, in keeping with the purposes herein. One manufacture of a test strip, including a reactive pad, similar to the test strip 100 of FIG. 1, is disclosed, for example, in U.S. Pat. No. 4,786,596. Of course, other compatible manufactures, in keeping with the purposes herein, can be employed.

In a specific embodiment, the target analyte may be alcohol and the target analyte specific oxidase may be an alcohol oxidase. The concentration of ethanol in breast milk may be determined by the reaction:

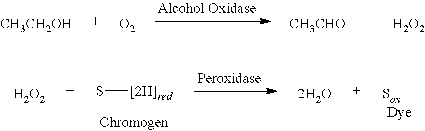

A number of colorimetric variants of alcohol oxidase-peroxidase method may be suitable for use in which different chromgens are used. ARTS (2,2′-azinobis(3-ethylbenzothiazoline-6-sulfonic acid)), a mixture of 4-aminoantipyrine (AAP) with 4-hydroxybenzosulfonate, or mixture of AAP with phenol. Similar method for quantitative assay of alcohol in biological liquids may also be employed using a sensitive non-carcinogenic chromogen 3,3′,5,5′-tetramethylbenzidine (TMB).

In a specific embodiment, the concentration of ethanol in breast milk may be determined by using alcohol oxidase-peroxidase in the presence of iodine indicator. The oxidant in the fluid sample reacts with iodide ion to form iodine. The iodine then is available to complex with a water-soluble polymer that also is present in the indicator reagent composition. Therefore, the indicator reagent composition contains an iodide salt, and typically potassium iodide. However, any water-soluble iodide salt having a cation that does not interfere with the assay for an oxidant can be used. Examples of other iodide salts are sodium iodide and lithium iodide. The iodine-polymer complex undergoes a color transition through various detectable and measurable degrees and intensities of color such that the degree and intensity of the color transition can be correlated to the concentration of oxidant in a fluid sample. In accordance with another aspect of the invention, the indicator reagent composition undergoes a differentiable color transition at high oxidant concentrations. The iodide salt may be present in the indicator reagent composition in an amount of about 1% to about 4%, and preferably in an amount of about 1.5% to about 3%, by weight of the indicator reagent composition. In a specific aspect, the iodide ion is present in an amount of about 1.75% to about 2.75%, by weight of the indicator reagent composition.

In addition to an iodide salt, the indicator reagent composition contains a starch or a water-soluble polymer. The water-soluble polymer is a nonionic or anionic polymer, and in particular is a cellulose-based polymer. However, other water-soluble polymers, such as polyvinylpyrrolidone, also can be used as the polymer in a present indicator reagent composition. The starch or water-soluble polymer may be present in the indicator reagent composition in an amount of about 0.1% to about 5%, and specifically about 0.2% to about 3% by weight of the indicator reagent composition.

The water-soluble, cellulose-based polymers may be derivatives of cellulose where hydroxy groups on the sugar moiety of cellulose are modified with a short chain alkyl (i.e., C₁-C₄), alkyl alcohol, or alkyl carboxylic acid. Examples of some common cellulose modifications are replacing a portion of the hydroxy groups with methyl, hydroxymethyl, hydroxyethyl, hydroxyethylmethyl, hydroxypropyl, hydroxypropylmethyl, or carboxymethyl groups, for example.

The color intensity of an iodine-polymer complex is inversely proportional to the number of carbon atoms in the modifying moiety. For example, a cellulose-based polymer having hydroxyethyl groups forms a darker brown color than a cellulose-based polymer having a hydroxypropyl group. Accordingly, color intensity is related to the hydrophilicity of the polymer. For example, hydroxyethylcellulose is soluble only in water, but not in organic solvents, whereas hydroxypropylcellulose is soluble in both water or organic solvent. Carboxymethylcellulose is highly soluble in water, and forms dark brown color with iodine.

Examples of water-soluble cellulose-based polymers useful in the invention include, but are not limited to, methylcellulose, hydroxymethylcellulose, hydroxyethylcellulose, hydroxyethylmethylcellulose, hydroxypropylcellulose, hydroxypropylmethylcellulose, carboxymethylcellulose and salts thereof, hydroxybutylcellulose, cellulose acetate, carboxymethylhydroxyethylcellulose, hydroxybutylmethylcellulose, and mixtures thereof. As illustrated hereafter, a blend of two or more water-soluble cellualose-based polymers provides a more intense color transition in response to peroxide or chlorine, and gives a wider color response to oxidant concentrations.

In addition to cellulose-based polymers, other water-soluble polymers can be used in the invention. Such water-soluble polymers are nonionic or anionic in character. Examples of useful water-soluble polymers include, but are not limited to, polyvinylpyrrolidone, hydrolyzed polyvinylpyrrolidone, poly(vinyl alcohol), poly(vinyl acetate), vinyl acetatevinyl alcohol copolymers, polyvinyloxazolidone, polyvinylmethyloxazolidone, co-polymers of vinylpyrrolidone and a vinyl amide of .gamma.-amine butyric acid, polyacrylic acid polymers, polyacrylic acid copolymers, partially or fully neutralized salts of polyacrylic acid polymers and polyacrylic acid copolymers, poly(methacrylic acid), poly(methacrylamide), poly(N,N-dimethylacrylamide), poly(N-isopropylacrylamide), poly(N-acetamidoacrylamide), poly(N-acetamidomethacrylamide), acrylic interpolymers of polyacrylic acid with poly(methacrylic acid), polyacrylic acid with poly(methacrylamide), polyacrylic acid with methacrylic acid, polyacrylamide, copolymers of acrylamide, acrylamide/sodium acrylate copolymers, acrylate/acrylamide copolymers, acrylate/ammonium methacrylate copolymers, acrylate/diacetoneacrylamide copolymers, acrylic/acrylate copolymers, adipic acid/dimethylaminohydroxypropyl diethylenetriamine copolymers, ammonium acrylate copolymers, ammonium styrene/acrylate copolymers, ammonium vinyl acetate/acrylate copolymers, aminomethylpropanol acrylate/diacetoneacrylamide copolymers, aminomethylpropanediol acrylate/diacetoneacrylamide copolymers, butyl benzoic acid/phthalic anhydride/trimethyolethane copolymers, diethylene glycolamine/epichl-orohydrin/piperazine copolymers, ethylene/vinyl alcohol copolymers, ethyl esters of polyethylenimines, isopropyl ester of methyl vinyl ether/maleic anhydride copolymers, melamine/formaldehyde resin, methoxyethylene glycol/dodecyl glycol copolymers, octadecene/maleic anhydride copolymers, octylacrylamide/acrylate/butylaminoethyl methacrylate copolymers, octylacrylamide/acrylate copolymers, polyethylene glycol/dodecyl glycol copolymers, polyethyleneimrine, phthalic anhydride/glycerin/glycidyl decanoate copolymers, metal salts of polyacrylic acid, metal salts of methyl vinyl ether/maleic anhydride copolymers, vinylpyrrolidone/eicosene copolymers, vinylpyrrolidone/ethyl methacrylate/methacrylic acid copolymers, vinylpyrrolidone/hexadecene copolymers, vinylpyrrolidone/vinyl acetate copolymers, polyvinypyrrolidone/vinyl acetate/itaconic acid copolymers, sodium acrylate/vinyl alcohol copolymers, sodium polymethacrylate, sodium polystyrene sulfonate, sodium styrene/acrylate/polyethylene glycol-10 dimaleate copolymers, sodium styrene/polyethylene glycol-10 maleate/nonoxynol-10 maleate/acrylate copolymers, styrene/acrylamide copolymers, styrene/acrylate/ammonium methacrylate copolymers, styrene/maleic anhydride copolymers, styrene/polyvinyloxazolidone copolymers, urea formaldehyde polymers, urea/melamine/formaldehyde polymers, vinyl acetate/crotonic acid copolymers, vinyl alcohol copolymers, and mixtures thereof.

According to one specific embodiment of the invention, the potassium iodide reaction may be as follows:

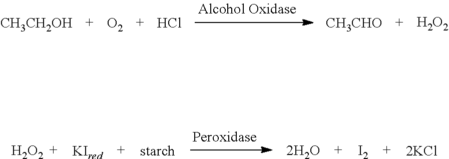

In an alternate embodiment, the potassium-iodide reaction described above, may be a single-enzyme system. That is, the reaction may be carried out without the presence of the peroxidase. For example, the reaction may be as follows:

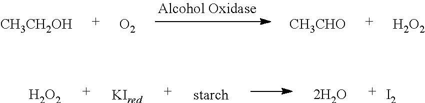

In another specific embodiment of the invention, a particular composition of the test reagent for the test strips and test kits of the invention is a dual enzyme system, which may include an alcohol oxidase enzyme extracted from yeast, together with a peroxidase and a hydrogen donor indicator, to provide an alcohol oxidase/peroxidase reaction. This composition reacts with ethyl alcohol if present in breast milk sample, to provide a resultant change of color in the reagent. The reaction is as follows:

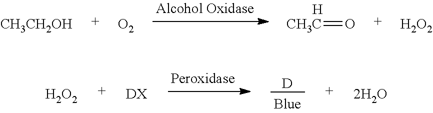

### Reagent Composition:

| | | |
|---|---|---|
| Tetramethylbenzidine | 0.176 | mg |
| Alcohol Oxidase (EC 1.1.3.13) | 0.5 | IU |
| Peroxidase (EC 1.11.1.7) | 30 | IU |
| Buffer | 0.757 | mg |
| Stabilizing Proteins | 0.190 | mg |

In the test reaction, the peroxidase functions as catalyst to induce a color change in the hydrogen donor and convert the hydrogen peroxide to water.

Isolated, recombinant, synthetic, and/or in vivo-produced anti-target analyte antibodies for diagnostic use in assaying a fluid sample for a target analyte of interest may be employed. Accordingly, methods of making and using such antibodies, including diagnostic and therapeutic compositions, methods, and devices are disclosed herein. Anti-target analyte antibodies useful in the methods and compositions of the invention are characterized by a high affinity binding to the target analyte present in the fluid sample (e.g., breast milk) with little or no cross-reactivity to other components present in the breast milk. The anti-target analyte antibodies of the invention are useful as a diagnostic marker for the presence of caffeine and its metabolites, nicotine and its metabolites, drugs of abuse and therapeutic drugs and its metabolites, alcohol, lactose, gluten, capsaicin, biomolecules, pathogens such as bacteria and viruses, and the like.

The methods for making the antibodies of the invention may include using one or more target analyte conjugates as an immunogen to stimulate an immune response. The methods include administering one or more target analyte conjugates to an animal using a suitable immunization protocol, and separating an appropriate antibody from a body fluid(s) of the animal, as described, for example, in Examples 1 and 2, infra. Alternatively, the target analyte conjugates may be used in phage display methods to select phage displaying on their surface an appropriate antibody, followed by separation of nucleic acid sequences encoding at least a variable domain region of an appropriate antibody. Phage display methods are well known to those of ordinary skill in the art. (See, for example, Antibody Phage Display; Methods in Molecular Biology, Vol. 178, O'Brien, Philippa M.; Aitken, Robert (Eds.) 2002)

The target analyte conjugates may be linked to a label to provide a detectable conjugate for use in receptor binding assays, such as immunoassays for the target analyte. Similarly, the anti-target analyte antibodies can be linked to a label to provide detectable anti-target analyte antibodies for use in receptor binding assays, such as immunoassays for the target analyte. The target analyte analogs can be linked to a label using methods well known to those skilled in the art. The anti-target analyte antibodies can be linked to a label using methods well known to those skilled in the art (for example, see, Immunochemical Protocols; Methods in Molecular Biology, Vol. 295, edited by R. Burns (2005)). The detectable target analyte conjugate or detectable anti-target analyte antibodies may be used in various homogenous, sandwich, competitive, or non-competitive assay formats, to generate a signal that is related to the presence or amount of the target analyte in a fluid sample.

In a specific embodiment, the immunoassay methodologies are competitive immunoassays for detection of anti-target analyte antibodies. The competitive immunoassay may be carried out in the following illustrative manner. A fluid sample, such as breast milk, potentially containing anti-target analyte antibodies, is contacted with one or more target analyte analogs conjugated to a solid support and with an anti-target analyte antibody conjugated to a detectable label. The anti-target analyte antibodies of interest, present in the sample, compete with the anti-target analyte antibody conjugated to a detectable label for binding with the one or more target analyte analogs conjugated to a solid support. In an alternative embodiment, the competitive immunoassay is carried out in the following illustrative manner. A fluid sample, such as breast milk, potentially containing anti-target analyte antibodies, is contacted with one or more target analyte conjugates linked to a detectable label and then with an antibody conjugated to a solid support. The anti-target analyte antibodies in the sample compete with the anti-target analyte antibodies on the solid support for binding with the target analyte conjugate linked to a detectable label. In either case, the signal obtained is inversely related to the amount of target analyte antibody of interest present in the sample.

According to one embodiment of the invention, breast milk may be screened for the presence of caffeine. In particular, immunoassays may be used to detect various analytes, including assays using anti-caffeine antibodies to detect the presence of caffeine. For example, enzyme-linked immunosorbent assays (ELISA) in which a caffeine-containing sample of breast milk is incubated in a vessel where it competes with peroxidase-labeled caffeine for the binding sites on caffeine antibodies followed by detection of a visible color change with the addition of o-phenylenediamine, for example.

The invention further provides diagnostic kits containing reagents for use in the immunoassay methods described above. Typically, such a kit contains at least one target analyte conjugate and/or at least one reagent that specifically binds to target analyte, such as an anti-target analyte antibody, as described below, of the invention. Kits typically also includes directions or instructions describing how to perform the above-described diagnostic assays, and/or how to interpret the results thereby obtained. In some kits, anti-target analyte antibodies are linked to an immobilized solid support and/or the target analyte conjugate is a target analyte analog immobilized on a solid support. The anti-target analyte antibody or target analyte conjugate may or may not be linked to an appropriate label.

The solid support for use in the methodology of the invention may include, without limitation, paper, sponge materials, cellulose, wood, woven and nonwoven fabrics, glass fiber, polymeric films, preformed and microporous membranes, synthetic and modified naturally-occurring polymers, or hydrophilic inorganic powders. Further, the solid support may be a test strip or other testing device and may include a support strip or handle, normally constructed from a hydrophobic plastic, and a reagent test region, containing a bibulous or a nonbibulous carrier matrix incorporating the anti-target analyte antibodies and/or the target analyte analogs of the invention. The carrier matrix may be an absorbent material that allows the test sample to move, in response to capillary forces, through the carrier matrix to contact the anti-target analyte antibody and/or target analyte analogs of the invention and produce a detectable or measurable signal, such as a color transition. The carrier matrix may be any substance capable of incorporating the anti-target analyte antibody and/or target analyte analogs of the invention, as long as the carrier matrix is substantially inert, and is porous or absorbent relative to the soluble components of the liquid test sample.

Accordingly, suitable antibodies for detecting a target analyte in a fluid sample such as breast milk include immunoglobulins, particularly IgEs, IgGs and IgMs, and particularly therapeutically or diagnostically relevant antibodies, including but not limited to, for example, antibodies to nicotine (and its metabolites), gluten, lactose, caffeine (and its metabolites), capsaicin, human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenyloin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g. respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gononrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like); and enzymes (and other proteins), including but not limited to, bacterial and viral enzymes such as HIV protease.

Further embodiments include other methodologies for detecting a target analyte in a fluid sample known by those of skill in the art. For example, target analytes may be detected by direct sensing method via chemical reaction. In this case, an assay may be developed such that the chemistry has a preferential bonding affinity with a target analyte, and results in target analyte detection without the use of antibodies or enzymes. In the this type of assay, the target analyte will specifically bond with the chemistry of the assay, thus changing the chemistry of the assay, and via a single or multiple stage reaction will produce a change observable by the consumer. One skilled in the art appreciates the various reagents that may be used in direct sensing methods, such as dye molecule indicators.

According to further embodiments, target analyte detection in a fluid sample may be carried out by various methods known in the art and the results of the target analyte detection may be observable by the end user by a variety of observable results, such as colorimetric transformation, color withdrawal, chemiluminescence transformation, or by digital electronic read out. In color presentation, a result is produced where an observation is from colorless to color. In color withdrawal, the results are observable by the end user as a change from color to colorless. The results may also be viewable by the user via a digital/electronic read out. For example, a reaction(s) may take place where the oxidation/reduction of certain chemistries result in the transfer of electrons and facilitate a change in electrical properties, such as current and/or voltage. Similar to the operation of a conventional blood glucose monitor, chemistries can be prepared that exhibit a flow of electrons (current) that can be monitored with a handheld device that amplifies the signal and uses a software counterpart to convert this data into a value known and understood to the end user by a display device or other type of output.

### Device Architecture

For illustrative purposes and convenience, the device and the testing procedure described below will be described using an ethanol testing reagent and testing for the presence of ethanol in breast milk. However, in no manner should the device or testing procedure be construed to be limited for solely testing for ethanol in breast milk. The device should be broadly construed and easily adapted by those of skill in the art to be able to test for any of the target analytes broadly described herein using any testing reagent or other device capable of detecting a target analyte.

Referring to FIG. 1, a test strip 100 comprises a reactive agent 102 maintained on a carrier strip 104. The carrier strip 104 has a handling portion 104a and a sampling portion 104b. The handling portion 104a of the carrier strip 104 is for human handling of the test strip 100 via that portion. The indicator agent 102 is connected on, impregnated in, or otherwise maintained at the sampling portion 104b of the carrier strip 104.

The carrier strip 104 is a paper, plastic, wood, or similar material. The carrier strip 104 is suitable for maintaining the indicator agent 102 of the test strip 100 and also for non-interference and non-reaction in testing operations utilizing the test strip 100 as later described herein. The test strip 100 can be slim and linear, as in FIG. 1, or can have other shape that generally provides a portion for handling by a human user and another portion for location of an agent for testing. For example, a stick or round pole structure, a triangular or square slip, or other dimensioned piece can be suitable. In many (but not necessarily all) instances, the agent's properties for testing can be affected or otherwise disturbed if allowed to touch skin, become exposed to anything other than liquids in testing, such as, for example, exposure to air, gases, water, sweat, oils, emulsions, non-test liquids, and/or be subjected to certain temperature, pressure or other extremes; therefore, human handling is usually best limited to the handling portion 104a of the carrier strip 104.

The reactive agent 102 may be an ethanol alcohol recognition composition or material. For example, the reactive agent 102 is reactive with ethanol alcohol if and when the reactive agent 102 is exposed to the alcohol in testing operations. Any of a variety of compounds or materials can be suitable as the reactive agent 102. The reactive agent 102 is itself adhereable, impregnable, wickable, stainable, or otherwise connectable in, with or to the carrier strip 104 at the sampling portion 104b. Alternately, a glue, attacher, or sticky matter is employed to retain the reactive agent 102 with the carrier strip 104 at the sampling portion 104b, provided, such matter must not affect or alter reactivity of or tests with the reactive agent 102 and must not be itself be reactive inconsistent with the reactive agent 102.

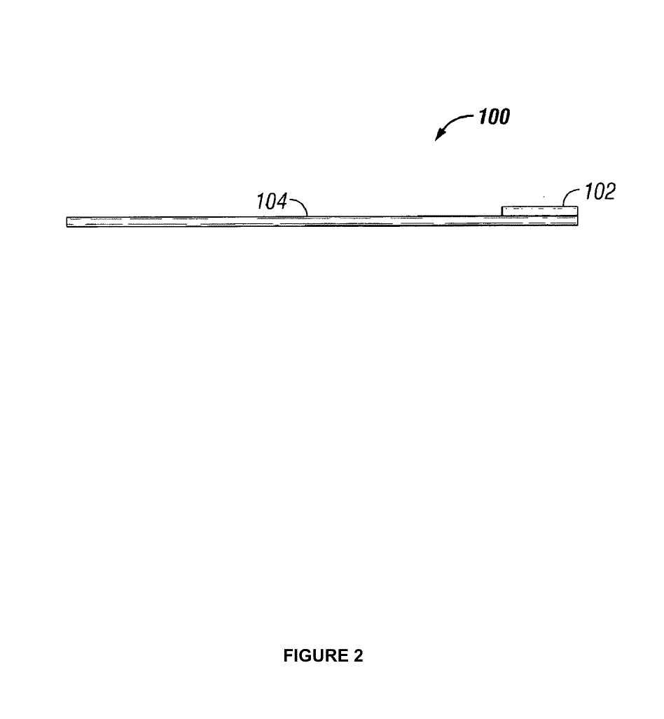

Referring to FIG. 2, the test strip 100 of FIG. 1 includes the carrier strip 104. The reactive agent 102 is maintained on a surface of the carrier strip 104. The reactive agent 102 is generally layered and attached to the carrier strip 104 at the sampling portion 104b.

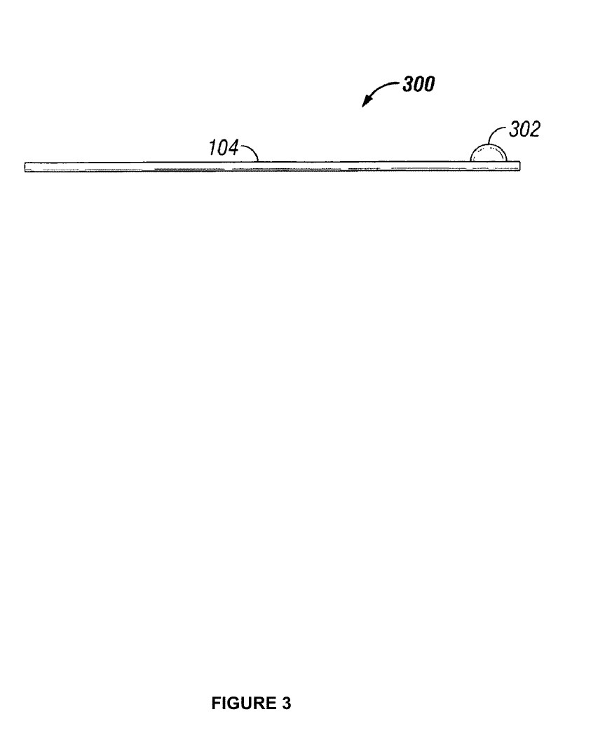

Referring to FIG. 3, an alternate test strip 300, includes the carrier strip 104, or similar support, to that of the test strip 100 of FIG. 1. An alternate reactive agent 302 is a drop of matter reactive to ethanol alcohol. The reactive agent 302 is similarly disposed at the sampling portion 104b of the carrier strip 104. The reactive agent 302 can be hardened material, affixed in, with or to the carrier strip 104, or otherwise thereon disposed, including in liquid, gel or other physical form.

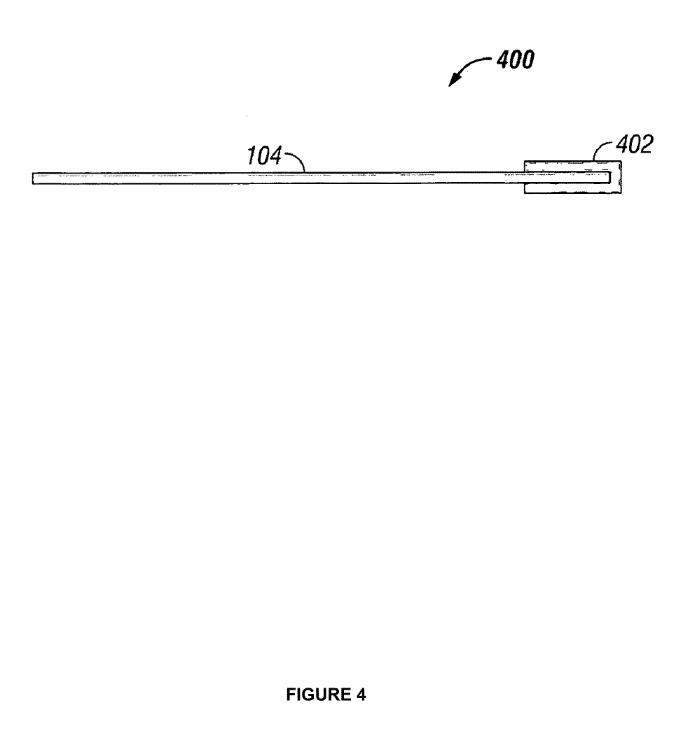

Referring to FIG. 4, another alternate test strip 400 also includes the carrier strip 104 or similar support. The carrier strip 104 has reactive agent 402 located on at least two sides of the carrier strip 104 at the sampling portion 104b. The sampling portion 104b is formed with the reactive agent 402, for example, by dipping the sampling portion 104b into the reactive agent 402, such as in manufacture where the reactive agent 402 is of a liquid form that hardens and attaches after dipping. Alternately, a cap-type form of reactive agent 402 can be attached to the sampling portion 104b at the end thereof. In FIG. 4, the reactive agent 402 is illustrated as formed on three sides at the sampling portion 104b (i.e., on a top side, a bottom side and an end); however, edges can also include the reactive agent 402, the reactive agent 402 can be disposed only on the top side and bottom side, or otherwise.

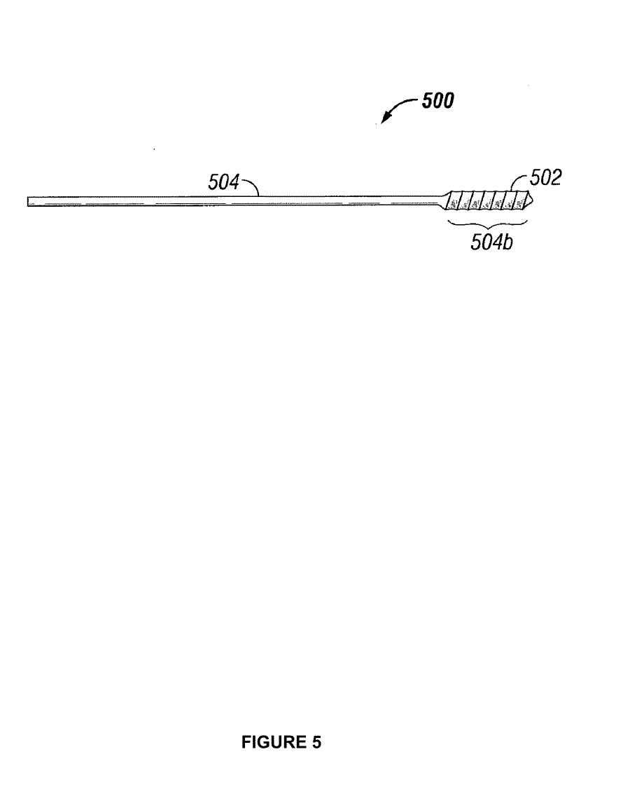

Referring to FIG. 5, another test strip 500 includes a carrier strip 504 that is a permeable, semi-permeable, wicking, blotting, a rigid or semi-rigid polymer, such as styrene, or other similar materials. The reactive agent 502 is contained within interstices of the material of the carrier strip 504 at the sampling portion 504b. The reactive agent 502 is liquid, dried liquid, powder, or hardened matter that is soaked up at least in part by the material of the carrier strip 504, for example, during manufacture, just prior to test initiation, or otherwise as may be applicable for the particular agent employed for testing.

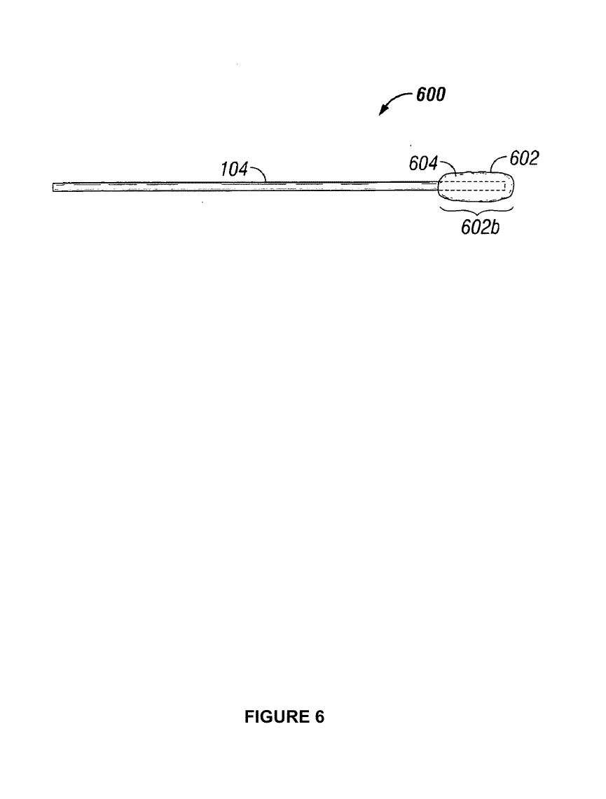

Referring to FIG. 6, another alternate test strip 600 includes the carrier strip 104, such as that of FIG. 1, and the carrier strip has an end carrier vehicle 602. The carrier vehicle 602 is a cotton, sponge, or similar material that can retain therein or thereon a reactive agent 604. The carrier vehicle 602 is formed, placed or joined to the carrier strip 104 at the sampling portion 602b. The reactive agent 604 is maintained by the carrier vehicle 602 with the test strip 600, for testing via the reactive agent 604 in the carrier vehicle 602.

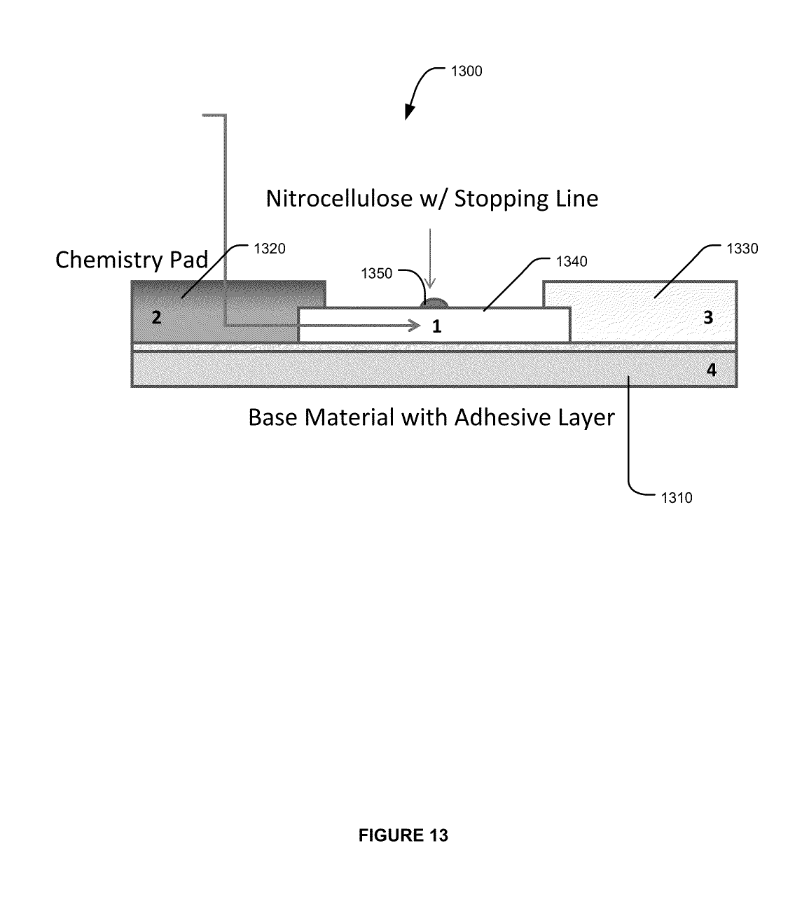

Referring to FIG. 13, another alternate test strip includes a lateral flow test strip 1300. Lateral flow test strip 1300 may include a base material with adhesive layer 1310, a chemistry pad 1320, a wicking pad 1330, an intermediate layer, such as a nitrocellulose filter paper 1340, and a stopping line 1350. As illustrated in FIG. 13, the nitrocellulose paper 1340 is disposed between the chemistry pad 1320 and the wicking pad 1330. A fluid sample is deposited onto the chemistry pad 1320. The chemistry pad includes at least one reagent capable of detecting the presence, absence, or concentration of the target analyte in the fluid sample. For example, the reagents may include enzymes, artificial receptors, antibodies, or indicator dyes, or other agents known in the art such as calcium indicator dyes. After the fluid sample is deposited onto the chemistry pad 1320, the fluid sample reacts with the reagents in the chemistry pad 1320, the fluid sample is pulled from the chemistry pad 1320 to region on the nitrocellulose paper 1340 to the stopping line 1340 via the wicking action of the testing strip, where the results would be indicated. Various reagents may be present at the stopping line 1340, and may prevent the fluid sample from traveling further up the testing strip. For example, these reagents may include surfactants, such as anionic surfactants which include without limitation, carboxylates, sulphonates, petroleum sulphonates, alkylbenzenesulphonates, naphthalenesulphonates, olefin sulphonates, alkyl sulphates, sulphates, sulphated natural oils and fats, sulphated esters, sulphated alkanolamides, alkylphenols, ethoxylated and sulphated; nonionic surfactants, ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides; cationic surfactants which include without limitation, quaternary ammonium salts, polyoxyethylene alkyl and alicyclic amines, 2-alkyl 1-hydroxyethyl 2-imidazolines; and amphoteric surfactants, amphoteric surfactants which include without limitation n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, and sodium salt.

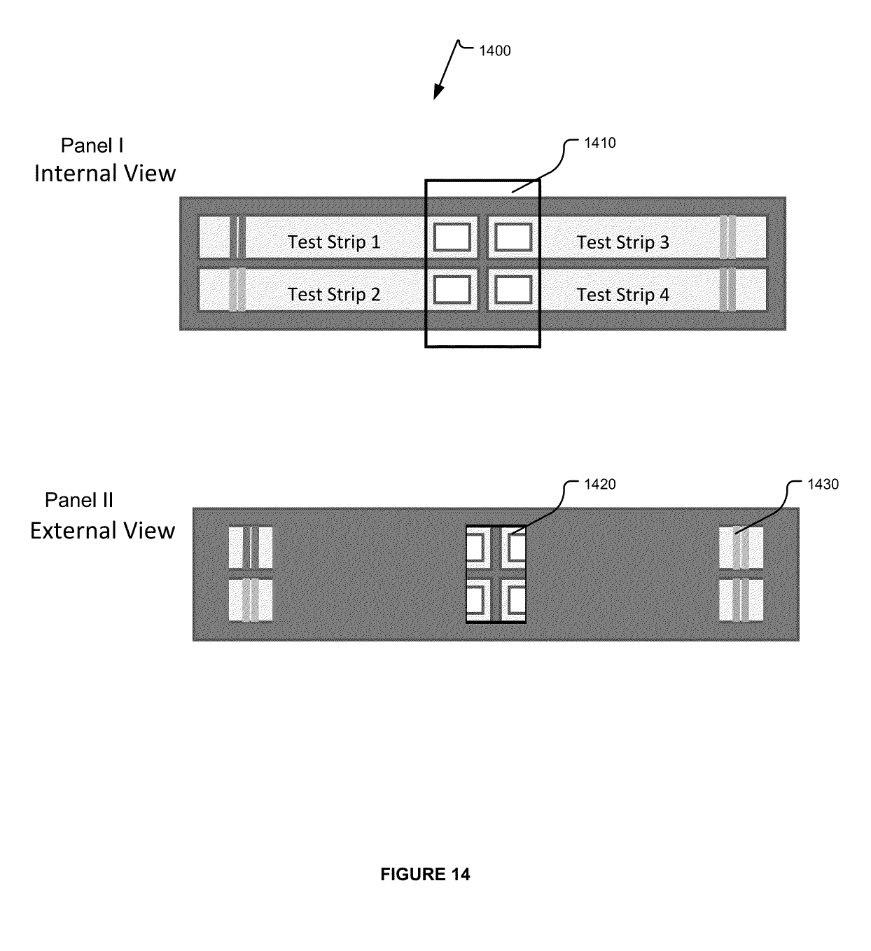

FIG. 14, Panel I and Panel II, illustrates an alternate embodiment, which shows multiple testing strips combined into one test enclosure for determining the presence of more than one target analyte in a single sample. FIG. 14, Panel I, shows an internal view of testing device 1400. By example, device 1400 may contain 4 individual test strips 1410. The number of test strip within the device is variable and may include any number of test strips such 2, 3, 4, 5, 6, 7, 8, 9, 10, etc. Each individual testing strip may have a structure similar to the device depicted in FIG. 13 and may be capable of detecting the presence, absence, or concentration of a different multiple target analytes in the fluid sample. The testing reagents may be present in the sampling region 1410 or throughout the individual testing strips. The user would deposit the fluid sample onto the single area denoted by the black outline e.g., by directly expressing milk fro the breast onto the sample region 1410 or by transferring milk that already has been expressed, e.g., by pumping. FIG. 14, Panel II shows an external view of device 1400. Panel II illustrates device 1400, with sample region 1420 and a viewable results region 1430. The viewable results region 1430 may also include stopping reagents that prevent any further migration of the fluid sample up the testing strip. The stopping reagents may include any of the above described surfactants. The wicking properties of the individual testing strips 1410 may pull the fluid sample from the sampling region to the result region 1430, where the results would be viewable by the user. Device 1400 includes at least one reagent capable of detecting the presence, absence, or concentration of at least one target analyte and preferably multiple target analytes in the fluid sample. For example, the reagents may include enzymes, artificial receptors, antibodies, or indicator dyes, such as calcium indicator dyes.

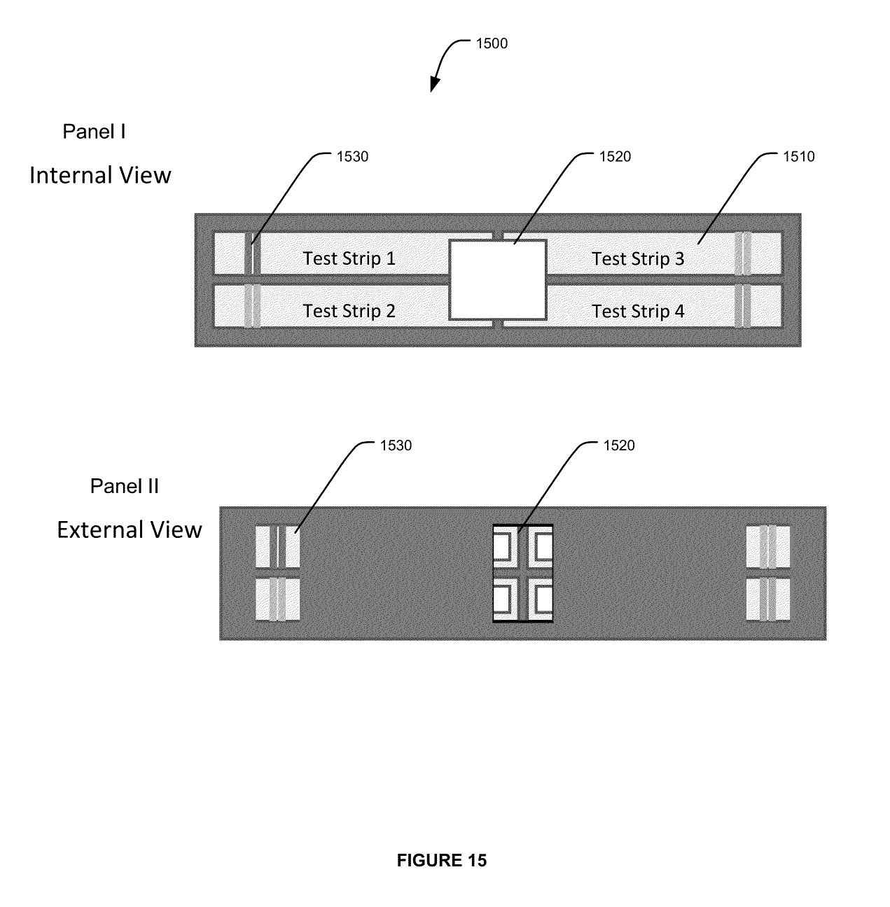

Another alternate embodiment is illustrated by FIG. 15, Panels I and II, which shows testing device 1500. Device 1500 functions in a similar fashion to device 1400 with the only difference being the use of a common sampling pad 1520, instead of discrete sampling pads as in the embodiment encompassed by FIG. 14. Each test strip 1-4 draws a sample fluid from the common sampling pad 1520.

In general, the testing substrate materials are chosen such that they enable the time to result and specificity described herein. In addition, the materials must allow the assay to proceed effectively when the liquid sample includes solution attributes that are likely to be present in breast milk and may interfere with the integrity of the testing results, such as fats, proteins, lactose, oligosacchrides, white blood cells, lactoferrin, lipase, probiotics, lyzozyme, lymphocytes, mast cells, immunoglobulins (e.g., IgG, IgA and IgM), interleukin, skin flora, and GI flora. In specific aspects, and depending upon on the specific target analyte being detected, the test strips and results may need to take into account base lines concentration of specific components found in breast milk.

Moreover, these substances, described above, may interfere with the accuracies obtained via the testing and test strips/kits herein. Additionally, because it is expected that non-laboratory trained individuals may be performing the testing with the test strips/kits, subjective determinations by those individuals of results can be varied. In general, with the particular test reagents described herein, the following substances in addition to the component present in breast milk, for example, may affect integrity of test results: peroxidases, strong oxidizers, reducing agents (e.g., ascorbic acid, tannic acid, pyrogallol, mercaptans and tosylates), billirubin, L-dopa, L-methyldopa, metampyrone, acetaminophen, acetylsalicylic acid, ampicillin, caffeine, ibuprofen, loratadine, sorbitol, DMSO, glycerol, and others. Further, other particular medications, diets, metabolism and other individualized characteristics of the breast-feeding mother can potentially affect results. Generally, however, for the purposes of most mothers, the testing, test strips, test kits and other features and aspects herein will generally provide a fair indication of alcohol in breast milk lactate, to allow those mothers to decide if there is any concern with feeding the breast milk so tested to infants.

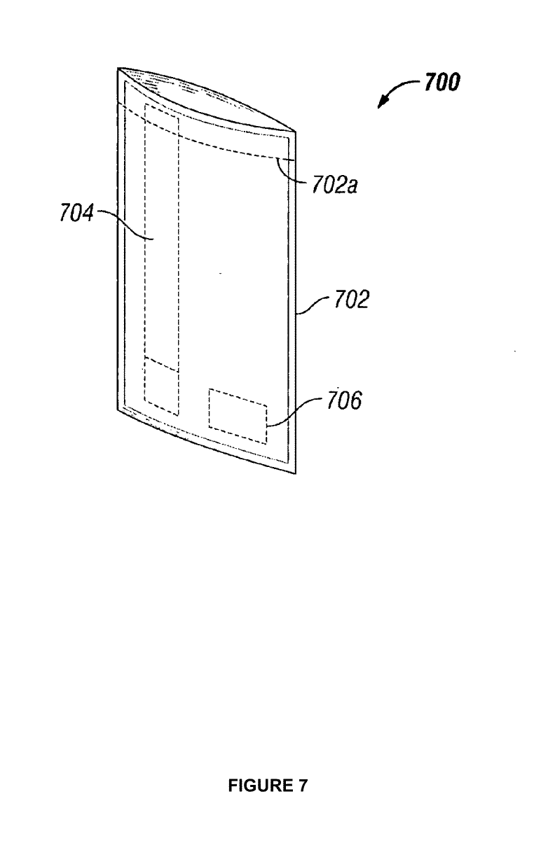

FIG. 7 illustrates a particular embodiment of the invention. In FIG. 7, a test kit 700 includes a package 702. The package 702 is a sealed, tearable enclosure, sufficient for containing a test kit strip 704 (substantially of the form of the test strips previously described, including a carrier strip and reactive agent). The package 702 is, for example, an environmentally sealed envelope, such as a metallic, plastic, paper or otherwise impermeable or semi-permeable material. The package 702 can be vacuum sealed or otherwise enclosed within a sterile (or, in certain instances, at least clean and generally clear lab or manufacturing facility) environment, wherein the test kit strip 704 is included prior to complete enclosure. The package 702 can, but need not necessarily, include tear dimples or other similar packaging aspects that enable ease of tearing or cutting to remove the test kit strip 704. In every event, the test kit strip 704 is protected by the package 702, once enclosed therein, from external matter that could contaminate, complicate, or otherwise affect testing via the test kit strip 704. The package 702 can further include other elements within the package 702, as desired, for example, desiccant 706, other matters to aid preservation or veracity, testing instructions, contents descriptions, regulatory or other warnings and notices, and other matters.

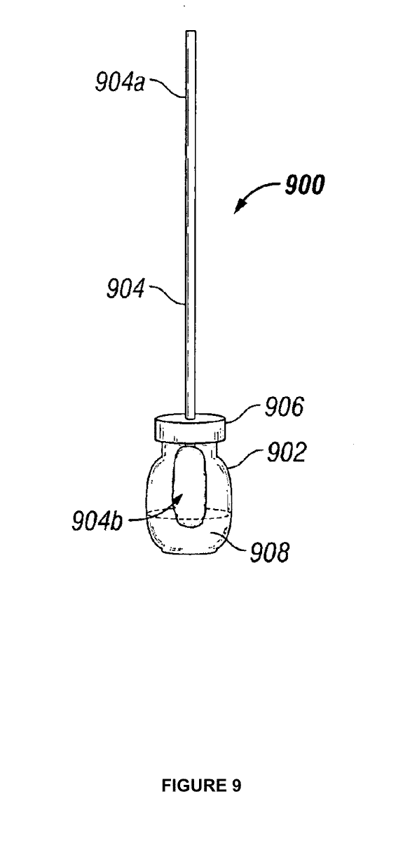

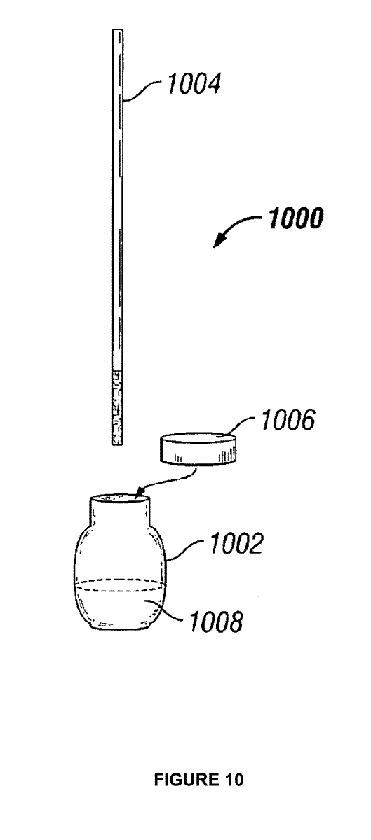

In use, the package 702 is opened, the test kit strip 704 is retrieved therefrom, and testing via the test kit strip 704 can be conducted. The package 702 is a single-use package which can be discarded. In other embodiments, package 702 may take the form of a multiple-use container, e.g., a re-usable bottle as shown in FIGS. 9-10, in which the test device is completely contained, as shown in FIG. 7. Alternatively, the multiple-use container could take the form of a re-usable package having a re-sealable enclosure, such a ziplock, provided a suitable amount of desiccant, such as 706 is provided.

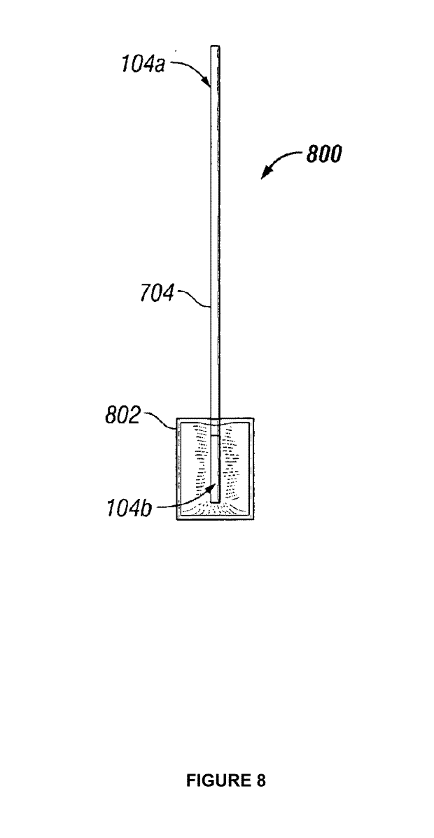

Referring to FIG. 8, an alternate test kit 800 includes an alternate package 802 and the test kit strip 704. A sampling portion 104b (substantially as described with respect to the other Figures) of the test kit strip 704 is enclosed within the package 802, and the handling portion 104a thereof protrudes through the enclosure of the package 802. For example, the package 802 is shrink-wrapped or formed around the sampling portion 104b, in a manner permitting the handling portion 104a (or part thereof) to extend outside the package 802. In the package 802, the enclosure is at least fairly completely sealed against or formed to or with the handling portion 104a, to prevent contamination or other testing concerns to reagent for testing of the sampling portion 104b. The package 802 can be folded back to expose the sampling portion 104b when testing is desired, or, as applicable, may be removed from the test kit strip 704.

Referring to FIG. 9, another alternate test kit 900 includes a vial 902. The vial 902 is sealed by a cap 906, such as a screw-on/off top or other sealing cap. A vial test strip 904 of the kit 900 includes a handling portion 904a and a sampling portion 904b. The test strip 904 is formed to extend through the cap 906 with minimal clearance sufficient to prevent exposure of contents of the vial 902 when the cap 906 is in place to seal the enclosure. Alternately, the test strip 904 is integral to the cap 906, and is either formed therewith, as part thereof, or is secured therewith extending through the cap 906. The handling portion 904a extends from the cap 906 outside the enclosure of the vial 902 and cap 906. Internally in that enclosure, the sampling portion 904b is disposed, extending from the cap 906 into the vial 902 (and, if and to the extent applicable, some length of extension of the handling portion 904a as may be necessary to cause the sampling portion 904b to sit in desired location within the vial 902). The vial 902 and cap 906 are each any of a glass, plastic, metal or similar rigid material sufficient to protect, and not react with, reagent of the sampling portion 904b.

In certain embodiments, a test reagent 908 (shown in phantom) can fill the vial 902, if either there is not reagent fixed at the sampling portion 904b (such as is the reactive agent 102, or similar, as previously discussed) or if the sampling portion 904b is capable of sitting/storing in a powder, liquid, or similar matter as the reagent material and collecting/retaining a sufficient portion of that reagent material when the sampling portion 904b is removed from the vial 902 for testing.

In use, the cap 906 is removed from the vial 902, and the sampling portion 904b with disposed reagent 908 (or, as applicable, reactive agent 102, etc.) is removed from in the vial 902. The user handles the handling portion 904a to remove the sampling portion 904b and conduct testing.

Referring to FIG. 10, another test kit 1000 includes a vial 1002 and cap 1006 to enclose a reagent 1008, for example, a liquid, powder, crystalline or other form. A test strip 1004 is external to the vial 1002 and cap 1006 enclosure, when testing is not performed. The test strip 1004 is that of the prior discussion and/or another design which allows capture of some of the reagent 1008 whenever the test strip 1004 is dipped into the reagent 1008 in the vial 1002. For example, the test strip 1004 can wick the reagent 1008 at an end or can attach or affix to reagent 1008 at an end of the strip 1004, for example, by glue, adherent, or similar means.

In use, the cap 1006 is removed from the vial 1002. The test strip 1004 is handled at an end opposite the intended testing end. The testing end of the test strip 1004 is then dipped into the open vial 1002 and into the reagent 1008. The test strip 1004 is removed from the vial 1002, with the reagent 1008 intact thereon at the testing end of the strip 1004. Testing can then proceed.

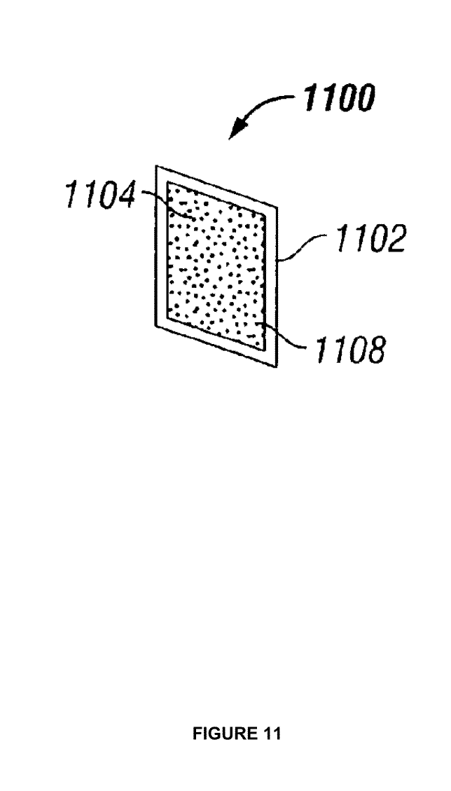

Referring to FIG. 11, another alternative test kit 1100 is an enclosed pouch 1102 with an internal space 1104 for storing a liquid, powder or similar content reagent 1108. The test kit 1100 need not, but can, include a test strip (similar to those previously described). In use, the pouch 1102 is opened, for example, by tearing or cutting, to allow the content reagent 1108 to be removed from the pouch 1102. The content reagent 1108 is then applied to a test sample. The content reagent 1108, for example, can be poured onto a surface or otherwise directly located on a sample for testing.

Alternately, the pouch 1102 can be semi-permeable to a sample for testing. In such alternative, the pouch 1102 can, itself, be placed into the sample for testing and/or the sample can be directed onto the pouch 1102. The pouch 1102 in such instance will necessarily either itself provide test readings or will allow viewing of readings shown by the contents.

### Testing Procedure

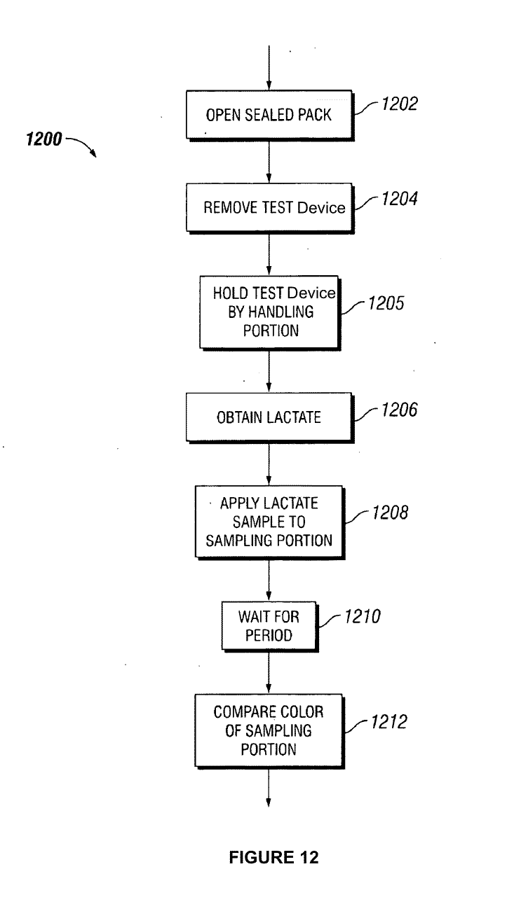

Referring to FIG. 12, a method 1200 of testing for ethanol alcohol in breast milk utilizes the foregoing test strips and test kits of the invention. The method 1200 commences with a step 1202 of opening a pack or enclosure containing the sampling portion of the test strip. As previously mentioned, the pack or enclosure protects integrity of the test strip/reagent for testing. On opening in the step 1202, the test strip/reagent is then useable for testing.

The test strip/sampling portion/reagent is removed from the pack or enclosure in a step 1204. The testing person handles the test strip at the handling portion, to prevent affects on test integrity. In a step 1204, the user continues handling the test strip by the handling portion during the testing method 1200.

A breast milk lactate from a breast-feeding mother is obtained in a step 1206. The quantity of the breast milk lactate obtained in the step 1206 need not be great, but should be sufficient for suitably wetting the reagent of the alcohol test and for yielding a proper result of the test. The breast milk lactate is applied, in a step 1208, preferably directly (but could also be indirectly, via a container of the breast milk or other specimen thereof), onto the reagent. For example, if using a test strip/test kit as herein described, the reagent pad at the sampling portion of the strip is wetted by breast milk sample.

A waiting period in a step 1210 must then be allowed, in order for the reaction to occur or not. For example, in testing with the test reagent particularly described above, a period of at least about 2 minutes is recommended. In this wait period, the reaction of the reagent with any alcohol in the breast milk takes place, and also the indicator is triggered therein to yield a visible result.

In a step 1212, the visible result is viewed and compared to detect any change indicative of alcohol presence in the breast milk lactate. If there is not any such indicative change, then there is usually little or no alcohol (neither ethanol alcohol or certain other alcohols, for example, alcohols of less than 5 carbons using the test reagent particularly described above). On the other hand, if there is an indicative change, then usually there is some presence of alcohol (ethanol or other, for example, alcohol of less than 5 carbons using the test reagent) in the breast milk lactate.

In alternatives, quantities of alcohol in breast milk can be estimated from the viewable results. For example, a more significant or pronounced indicative change can signal greater alcohol concentration in the breast milk; whereas, a less significant or less pronounced change can signal a lesser alcohol concentration of the milk. Data (for example, obtainable through other more exact and precise laboratory testing means than those described herein, as will be known to those skilled in the art) is corresponded with varied viewable results from different alcohol-breast milk concentrations employing the testing described herein, and can be compiled as average concentration readings, to provide charts, color tabs, or other comparative devices, for estimation of concentration levels based on the viewable results of the testing described herein.

Without further elaboration, it is believed that one skilled in the art using the preceding description can utilize the invention to the fullest extent. The following examples are illustrative only, and not limiting of the disclosure in any way whatsoever.

### EXAMPLES

### Specific Example 1

### Development of Anti-Caffeine Antibodies

Anti-caffeine monoclonal antibodies (MAb) are produced with regard to acceptance criteria that included high-affinity binding to caffeine, minimal cross-reactivity with the components that may be found in breast milk, isotypes that allow for cost-effective production of purified monoclonal antibodies, and production of hybridoma cell lines that will secrete high levels of MAb.

To synthesize caffeine immunogens and immunize mice, sera from immunized mice are screened for caffeine-binding antibodies by a competitive ELISA modeled on previously described protocols, e.g., as described in Fickling, S. A. et al., “Development of an Enzyme-linked Immunosorbent Assay for Caffeine,” J. Immunol. Meths., 129(2): 159-64 (1990). Briefly, BSA-caffeine conjugates or BSA-caffeine peptides are synthesized using standard methods, including the linkers DSS, EMCS-IT, and others. These methods are described, e.g., in Wong S S, Wong L J., “Chemical Crosslinking and the Stabilization of Proteins and Enzymes,” Enzyme Microb. Technol., 14(11):866-74 (1992); Mattson, G., et al., “A Practical Approach to Crosslinking,” Molecular Biology Reports, 17, 167-183 (1993); and Partis, M. D., et al. (1983), “Crosslinking of Proteins by omega-maleimido alkanoyl N-hydroxysuccinimide Esters,” J. Protein. Chem., 2, 263-277 (1983). The BSA-caffeine conjugates were used in ELISA according to standard ELISA protocols, e.g., as described in Harlow & Lane. Antibodies: A Laboratory Manual. Cold Spring Harbor Laboratory, Cold Spring Harbor, N.Y., 1988. BSA-caffeine conjugate are coated onto high protein-binding EIA plates, incubated with serum samples with and without the presence of free caffeine, and antibody binding is detected using alkaline-phosphatase-linked goat anti-mouse-immunoglobulin (Ig) antiserum, followed by incubation with p-nitrophenyl phosphate. Substrate conversion rate is measured by optical density at 405 nm. Anti-caffeine antibodies are identified by decreased binding to BSA-caffeine when free caffeine was present.

Hybridomas producing mAbs fitting these acceptance criteria are selected, expanded, and preserved in cryobanks mAbs for use in development of the caffeine test strips are purified from secretions of these hybridomas. After identification of mice with high titers of anti-caffeine antibodies, fusions are performed to isolate hybridomas secreting anti-caffeine antibodies. Such hybridomas are identified by screening hybridoma cell culture supernatants using the same competitive immunoassay.

The above methods should not be construed to be limited to producing anti-caffeine antibodies, but one skilled in the art would recognize that these known techniques could be used to generate antibodies against any desired target analyte.

### Specific Example 2

### Development of Anti-Target Analyte Antibodies

The immunization protocol for generating the anti-target analyte antibodies of the invention is carried out as follows:

Six California breed rabbits are immunized with a target analyte. Three of the six rabbits are immunized with target analyte conjugated with BSA and three other rabbits are immunized with target analyte conjugated with KLH. For primary immunizations, each rabbit is injected with 0.5 mg of the target analyte conjugate in 1 ml of phosphate buffered saline (PBS) mixed with 1 ml of Freund's complete adjuvant. Each rabbit receives 20-30 intradermal injections on their shaved back. Each rabbit is boosted with 0.25 mg of immunogen in 1 ml PBS mixed with equal volume of Freund's incomplete adjuvant in the hind legs. The boosting shots are given each month after the primary injection. Test bleeds of 5 ml blood are taken from each rabbit 7-10 days after each boost. Production bleeds of 40 ml are taken from each rabbit after the third booster shot, when the antisera titer is greater than about 1:2000.

The anti-SDMA antibodies are isolated and purified from the antisera.

### Specific Example 3

A test strip having a handling portion and a sampling portion, with a test reagent disposed on the sampling portion, was obtained, for example, from Chematics, Inc. P.O. Box 293, North Webster, Ind., USA 46555. A breast-feeding mother consumed a bloody mary and a glass of white wine at brunch during the period 11 am-1 pm. At about 4 pm that same day, a sample of breast milk lactate was taken from the mother's breast. The sample was disposed directly from the breast onto the reagent of the sampling portion. At least 2 minutes were waited for testing reactions to occur. The mother observed the test results, and the test results indicated no change in color of reagent. This indicated to the mother that there was no presence of ethanol alcohol (from the earlier consumption) in the breast milk at that time. The mother thereafter immediately again performed a second testing with a new, unused but identical test strip. The mother obtained a next sample of breast milk lactate from her breast. The sample was contacted with the reagent for a period of at least 2 minutes. The reagent of the second test strip did not change color, and the mother observed that the second test results also indicated lack of presence of ethanol alcohol in the breast milk at that time.

The same mother, three days thereafter, consumed a beer and two and a half glasses of wine during the period between 7 pm-10:30 pm of that day. At 12:30 am the next morning, the mother remaining awake during the period from 10:30 pm to 12:30 am, conducted a next test of her breast milk lactate. In the test, an identical, but new and unused, test strip, like those used in the prior tests, was employed. The mother contacted breast milk then obtained by her with the reagent. After a wait period of at least 2 minutes, the mother observed some darkening change in color of the reagent of the test strip. The mother observed that the test result indicated a presence of ethanol alcohol in her breast milk at that time. The mother compared the resulting color of the reagent to a comparison tab provided with the packaging of the test strip, also obtained from Chematics, Inc. (a sample of the comparison tab is included in Appendix A hereto). Based on the mother's comparative observance, the mother estimated that her breast milk did contain some alcohol, and the mother estimated that alcohol concentration of the breast milk to be about 0.3% per the comparison tab. The mother then slept until about 6 am. At 6 am, the mother next tested her lactate then obtained, using another identical, but new and unused, test strip. The mother wetted the reagent of the test strip with the breast milk. After at least 2 minutes, the mother observed the reagent of the test strip and observed no change in color, indicating to the mother the lack of presence of alcohol in her breast milk at that time.

The same mother, again, ten days thereafter, consumed alcoholic beverages. The mother did not account for the number or type of alcoholic drinks she consumed, however, the mother states she began drinking the beverages at about 7 pm that evening and continued casual drinking throughout the evening until about 1:30 am the next morning. The mother slept and woke to test at about 7 am. At 7 am, the mother obtained breast milk from her breast. The mother contacted the breast milk and the reagent of an identical, new and unused, test strip. The mother waited 2 minutes to observe the test results. The reagent color that was observed appeared dark, having changed color significantly. The mother observed that the test result indicated the presence of substantial alcohol in her breast milk at the time, on the order of about 0.3% or more. Later in the day, at about 10 am, the mother again conducted the same test in substantially the same manner. The result of the test continued to indicate presence of substantial alcohol in the mother's breast milk at that time. The mother's comparison to the comparison tab approximations indicated the breast milk contained on the order of about 0.3% alcohol. Thereafter, on the same day at about 2 pm, the mother again tested her breast milk in similar manner. The mother observed that the result of the test indicated lack of presence of alcohol in the mother's breast milk taken at that time, the reagent having not changed in color to give other/different indication.

Another breast-feeding mother also tested her breast milk lactate for alcohol presence. One evening, this breast-feeding mother consumed 4 glasses of wine at dinner during the period 7:30 pm-9:30 pm. At about 10:30 pm that same night, this mother tested her breast milk lactate. The sample was disposed directly from the breast onto the reagent of the sampling portion. At least 2 minutes were waited for testing reactions to occur. The mother observed the test results, and the test results indicated the breast milk contained on the order of about 0.3% alcohol. At 5 am the next morning, the mother again tested her breast milk in similar manner. The testing strip indicated to the mother that there was no presence of ethanol alcohol (from the earlier consumption) in the breast milk at that time.

The same mother, the next evening, consumed two and a half glasses of wine during the period between 8:30 pm-10:30 pm of that day. At 11 pm that same evening, the mother contacted breast milk then obtained by her with the reagent. The mother observed that the test resulted in a presence of ethanol alcohol in her breast milk contained on the order of about 0.3% alcohol. To show a comparison with the concentration of alcohol in her breast milk, at that same time, a saliva test was also done, indicating her blood alcohol level was 0.08%.

To compare her results in a separate sequence of testing, one evening 3 weeks later, this breast-feeding mother consumed 2 martinis and 2 beers during the period 7 pm-10 pm. At about 12:00 am that same night, this mother pumped and tested her breast milk lactate. The sample was disposed directly from the breast onto the reagent of the sampling portion. At least 2 minutes were waited for testing reactions to occur. The mother observed the test results, and the test results indicated the breast milk contained on the order of about 0.08% alcohol. The next evening, the mother again consumed alcoholic beverages in the form of 4 beers during the period 5 pm-7 pm. Two hours later, at 9 pm, she then tested her breast milk in a similar manner as before. The testing strip indicated to the mother that there was a presence of ethanol alcohol (from the earlier consumption) in the breast milk at that time, indicating a level of 0.3%.

Other alternatives are possible in keeping with the foregoing and all such alternatives are included herein. For example, different support or carriers can be employed for the test strip or other device. Also, the particular active reagent for testing can be selected from among the various alternatives, in keeping with the purposes herein. Test strips and test kits can have a variety of forms/styles, depending on desired aspects. Comparison tabs or other color or indicator estimation measures can be derived and utilized. Additionally, the test strips, kits, and methods can be employed solely or primarily for purposes of detecting a presence of a target analyte in breast milk lactate, versus obtaining an estimated concentration. It is expected that with certain mothers, given wide variation in lifestyles, diets, metabolism, activities, and consumption, there are certain instances that may be more or less susceptible to obtaining accurate results of the breast milk-target analyte testing. Further, certain drugs, medications, and environments could impact test results, either adversely or beneficially. Variations in reagent compositions, as well as particular components of the compositions, can yield different results. Multiple testing via a varied assortment of such compositions could, for example, be conducted in each testing instance, in order to obtain more verifiable outcomes. Of course, as has been described herein with several examples and embodiments, more generalized concepts herein can be employed in testing for presence of target analytes in mothers' breast milk under differing and other conditions.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in cellular and molecular biology, chemistry, or in the relevant fields are intended to be within the scope of the appended claims.

The disclosures of all references and publications cited above are expressly incorporated by reference in their entireties to the same extent as if each were incorporated by reference individually.

What is claimed is:

1-28. (canceled)

29. A method of protecting a nursing infant from exposure to a target analyte present in breast milk that may have adverse effects on the nursing infant, said method comprising the steps of:
obtaining a breast milk sample from a subject, wherein the subject is suspected of ingesting a target analyte that may be harmful to the nursing infant;
detecting the presence, absence, or concentration of the target analyte in the breast milk by detecting a change in at least one reagent; and
if the change indicates the presence of the target analyte or concentration of the target analyte above a predetermined level, waiting a period of time to allow for clearance of the substance from the breast milk prior to breast feeding.

30. The method of claim 29, wherein the change comprises one of an observable colorimetric change or a readout indicative of a change in electrical properties of the detecting reagent.

31. (canceled)

32. The method of claim 29, wherein said at least one reagent comprises a reagent for performing a single or multiple enzyme test.

33. The method of claim 29, wherein the target analyte is selected from the group consisting of lactose, gluten, nicotine, triglycerides, drugs of abuse, therapeutic drugs, capsaicin, and alcohol.

34. A method of determine if a lactating mother is afflicted with mastitis, said method comprising the steps of:
providing a test device including a sampling portion, and one or more reagents that can detect a target analyte associated with mastitis disposed on the sampling portion;
contacting the one or more reagents on the sampling portion with the breast milk lactate for a predetermined period of time to allow any target analytes associated with mastitis present in the breast milk lactate to react with the test reagent and induce a detectable change in the test reagent; and
observing a result after the predetermined period of time has lapsed wherein a detectable change in the target analyte test reagent is indicative of mastitis.

35. The method of claim 34, wherein the detectable change in the test reagent is a color change.

36. (canceled)

37. (canceled)

38. The method of claim 35, wherein the concentration of the target analyte associated with mastitis is estimated in the breast milk lactate by comparing the visible color change in the test reagent to a viewable concentration standard device.

39. (canceled)

40. (canceled)

41. The method of claim 34, wherein the target analyte associated with mastitis is selected from the group consisting of white blood cells, sodium, inflammatory cytokines, and bacterial waste products.

42. A method of testing breast milk lactate for the presence of alcohol, said method comprising the steps of:
providing a test strip including at least one of a sampling portion; a single chemistry portion, or a multiple chemistry portion; and an alcohol test reagent disposed on at least one of the sampling portion, the single chemistry portion, or the multiple chemistry portion;
contacting the alcohol test reagent on the sampling portion with the breast milk lactate for a predetermined period of time to allow any alcohol present in the breast milk lactate to react with the alcohol test reagent and induce a detectable change in the alcohol test reagent; and
observing a result after the predetermined period of time has lapsed wherein a detectable change in the alcohol test reagent is indicative of the presence of alcohol in the breast milk lactate.

43. The method of claim 42, wherein the detectable change in the alcohol test reagent is a color change.

44. The method of claim 43, wherein the alcohol concentration is estimated in the breast milk lactate by comparing the visible color change in the alcohol test reagent to a viewable concentration standard device.

45. The method of claim 44, wherein the viewable concentration standard device comprises a card having a plurality of color tabs such that each of the plurality of color tabs is a different color corresponding to a different alcohol concentration level.

46. (canceled)

47. (canceled)

48. The method of claim 42, wherein the sampling portion includes a carrier vehicle that retains the alcohol test reagent.

49. The method of claim 48, wherein the carrier vehicle is fabricated from cotton.

50. The method of claim 42, wherein the alcohol test reagent in said contacting step comprises a hydrogen donor indicator, an alcohol oxidase, and a peroxidase.

51. The method of claim 50, wherein the hydrogen donor is selected from the group consisting of ABTS (2,2′-azinobis(3-ethylbenzothiazoline-6-sulfonic acid)), a mixture of 4-aminoantipyrine (AAP) with 4-hydroxybenzosulfonate, a mixture of AAP with phenol, 3,3′,5,5′-tetramethylbenzidine (TMB) tetramethylbenzidine, and a mixture of potassium iodide and starch.

52. The method of claim 50, wherein the hydrogen donor is a mixture of potassium iodide and starch.

53. The method of claim 42, wherein the predetermined period of time in said contacting step is about 2 minutes.

54. The method of claim 42, wherein the test strip is for one-time general consumer use and is disposable.

55. The method of claim 42, wherein said test device further comprises a handling portion configured to be contacted by an individual without contacting the sampling portion.
